(19) United States
(12) Patent Application Publication
(10) Pub. No.: US 2026/0305947 A1
(43) Pub. Date: Oct. 8, 2026

(54) POLARIZATION SENSITIVE BIOMETRIC MONITORING

(71) Applicant: Meta Platforms Technologies, LLC, Menlo Park, CA (US)

(72) Inventors: Mantas Zurauskas, Redmond, WA (US); Robert Dale Cavin, Bellevue, WA (US)

(21) Appl. No.: 19/569,332

(22) Filed: Mar. 17, 2026

Related U.S. Application Data
(60) Provisional application No. 63/783,518, filed on Apr. 4, 2025.

(51) Int. Cl. A61B 5/00

(52) U.S. Cl. CPC A61B 5/0059; A61B 5/6803; A61B 5/7278

(57) ABSTRACT

The disclosed method may include capturing a light reflected from a skin and detecting a polarized light signal from the captured reflected light. The method may also include analyzing the detected polarized light signal, detecting a specular light component and/or a diffused light component from the polarized light signal, and determining a biometric measurement based on the analyzing the specular light component and/or the diffused light component. Various other methods, systems, and computer-readable media are also disclosed.

### CROSS REFERENCE TO RELATED APPLICATION

This application claims the benefit of U.S. Provisional Application No. 63/783,518, filed Apr. 4, 2025, the disclosure of which is incorporated, in its entirety, by this reference.

### BRIEF DESCRIPTION OF THE DRAWINGS

The accompanying drawings illustrate a number of example embodiments and are a part of the specification. Together with the following description, these drawings demonstrate and explain various principles of the present disclosure.

FIGS. 1A-B illustrates examples of unfiltered and filtered images of skin.

FIG. 2 is a simplified diagram of an example system for polarization sensitive biometric monitoring.

FIG. 3 is a flow diagram of polarization sensitive biometric monitoring.

FIG. 4 is a simplified diagram of an example glasses device for polarization sensitive biometric monitoring.

FIG. 5 is a simplified diagram of an example wrist wearable device for polarization sensitive biometric monitoring.

FIGS. 6A-B illustrate simplified diagrams of example polarizer configurations for sensors.

FIG. 7 is a flow diagram of an example process for polarization sensitive biometric monitoring of different biometric parameters.

FIG. 8 is a flow diagram of an example method for polarization sensitive biometric monitoring.

FIG. 9 is an illustration of an example artificial-reality system according to some embodiments of this disclosure.

FIG. 10 is an illustration of an example artificial-reality system with a handheld device according to some embodiments of this disclosure.

FIG. 11A is an illustration of example user interactions within an artificial-reality system according to some embodiments of this disclosure.

FIG. 11B is an illustration of example user interactions within an artificial-reality system according to some embodiments of this disclosure.

FIG. 12A is an illustration of example user interactions within an artificial-reality system according to some embodiments of this disclosure.

FIG. 12B is an illustration of example user interactions within an artificial-reality system according to some embodiments of this disclosure.

FIG. 13 is an illustration of an example wrist-wearable device of an artificial-reality system according to some embodiments of this disclosure.

FIG. 14 is an illustration of an example wearable artificial-reality system according to some embodiments of this disclosure.

FIG. 15 is an illustration of an example augmented-reality system according to some embodiments of this disclosure.

FIG. 16A is an illustration of an example virtual-reality system according to some embodiments of this disclosure.

FIG. 16B is an illustration of another perspective of the virtual-reality systems shown in FIG. 16A.

FIG. 17 is a block diagram showing system components of example artificial- and virtual-reality systems.

FIG. 18 an illustration of an example system that incorporates an eye-tracking subsystem capable of tracking a user’s eye(s).

FIG. 19 is a more detailed illustration of various aspects of the eye-tracking subsystem illustrated in FIG. 18.

Throughout the drawings, identical reference characters and descriptions indicate similar, but not necessarily identical, elements. While the example embodiments described herein are susceptible to various modifications and alternative forms, specific embodiments have been shown by way of example in the drawings and will be described in detail herein. However, the example embodiments described herein are not intended to be limited to the particular forms disclosed. Rather, the present disclosure covers all modifications, equivalents, and alternatives falling within the scope of the appended claims.

### DETAILED DESCRIPTION OF EXAMPLE EMBODIMENTS

IPPG (Photoplethysmography) is a non-invasive optical technique that monitors heart rate by detecting changes in blood volume within the tissue’s microvascular bed. IPPG employs optical sensors like cameras or photodetectors in conjunction with active illumination. However, a significant challenge is the specular reflections from wet or oily skin and makeup, which can rapidly alter and negatively impact the signal-to-noise ratio (SNR) and the system’s sensitivity.

The present disclosure provides a polarization-sensitive optical heart rate detection system that enhances robustness in tracking heart rates. By utilizing both polarized illumination and polarization-sensitive detection, the systems and methods provided herein may effectively separate the specular reflection from the diffuse component. This separation may not only improve heart rate monitoring but also may allow for the assessment of skin hydration and sweat levels.

The present disclosure is generally directed to a polarization sensitive skin monitoring and PPG system. As will be explained in greater detail below, embodiments of the present disclosure may include an optical sensor having a polarizer, and configured to take a biometric measurement based on capturing diffused light from skin over time.

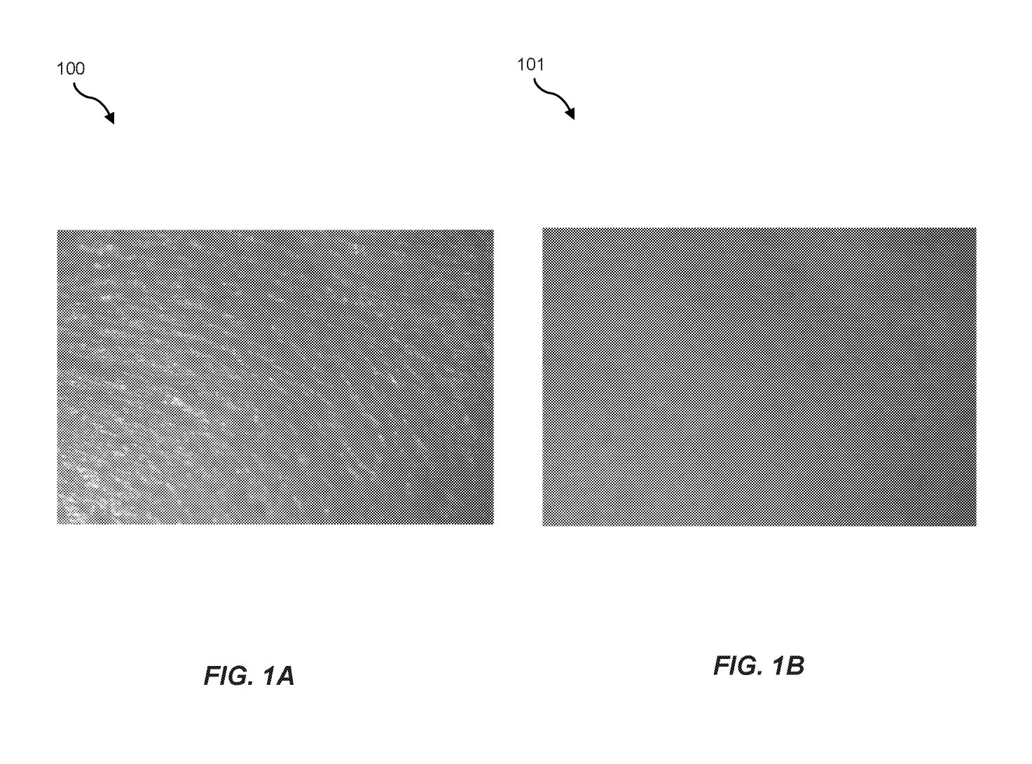

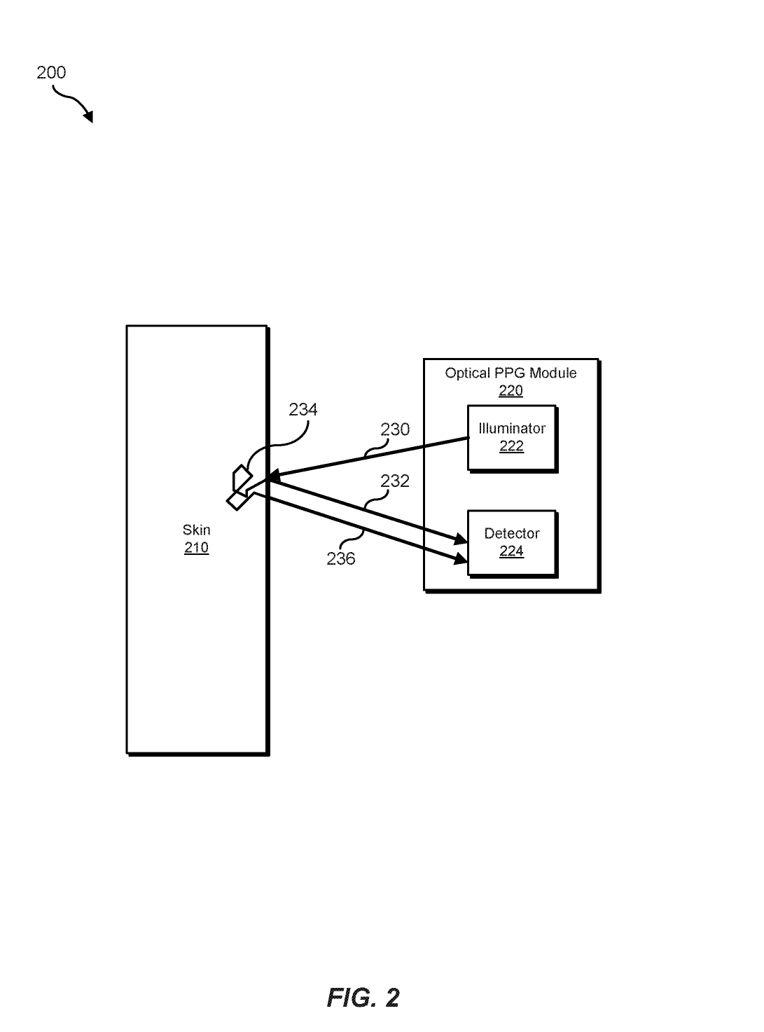

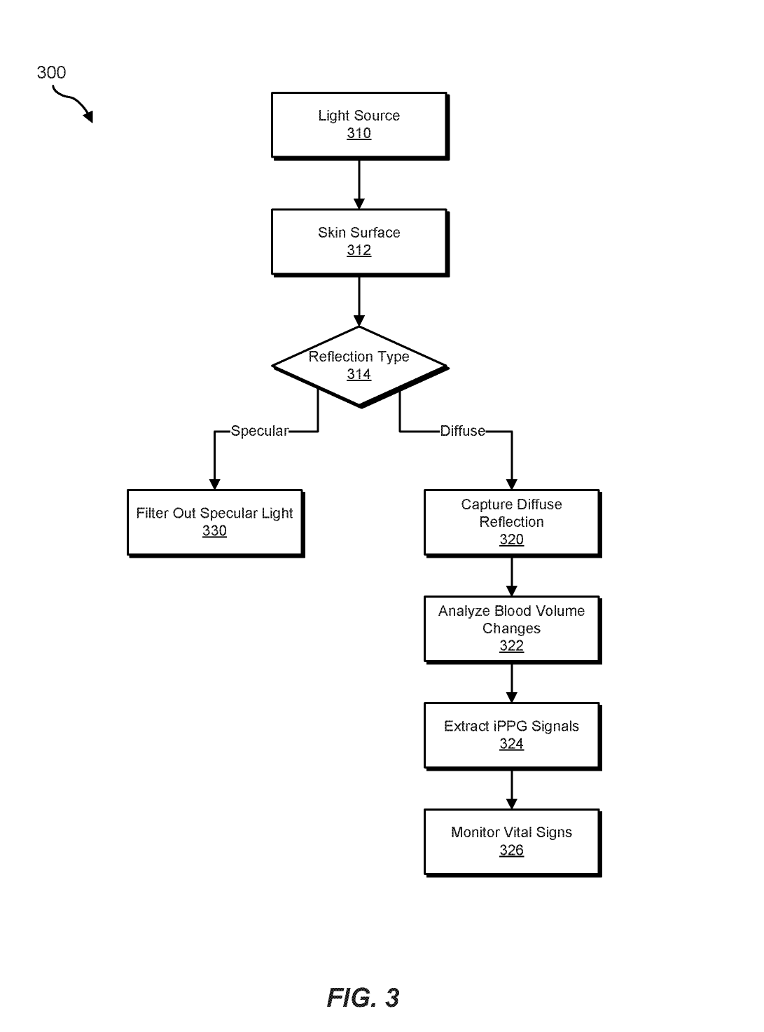

The following will provide, with reference to FIGS. 1-8, detailed descriptions of polarization sensitive biometric monitoring. Detailed descriptions of polarization filtering for skin will be provided in connection with FIG. 1A-1B. Detailed descriptions of example devices for polarization sensitive biometric monitoring will be provided in connection with FIGS. 2, 4, 5, and 6A-6B. Detailed descriptions of examples processes for polarization sensitive biometric monitoring will be provided in connection with FIGS. 3, 7, and 8.

Features from any of the embodiments described herein may be used in combination with one another in accordance with the general principles described herein. These and other embodiments, features, and advantages will be more fully understood upon reading the following detailed description in conjunction with the accompanying drawings and claims.

FIG. 1 illustrates an unfiltered image 100 and a filtered image 101 of skin. Both images were captured with a polarization camera and using linearly polarized LED illumination on the skin. Unfiltered image 100 was obtained by averaging values from four polarization sensitive sub-pixels. Specular light filtering for filtered image 101 was performed by selecting min value from a group of four polarization sensitive sub-pixels.

In the context of imaging photoplethysmography (iPPG), which is a non-invasive method used to detect blood volume changes in the microvascular bed of tissue, specular light is generally not necessarily helpful due to several reasons, including reflection interference, signal-to-noise ratio (SNR), consistency of measurement, and focus on diffuse measurement.

Regarding reflection interference, specular light reflects off the skin surface and does not penetrate into deeper layers where blood volume changes occur. This surface reflection can mask the subtle variations in light absorption caused by the pulsatile changes in blood volume, which are crucial for accurate iPPG measurements.

Regarding SNR, specular reflections can significantly increase the noise in the captured signal. Since iPPG relies on detecting very small changes in light intensity related to blood flow, any additional noise can make it difficult to isolate the true physiological signals from the noise caused by reflections.

Regarding consistency of measurement, specular reflections can vary significantly depending on the angle of incidence of the light and the properties of the skin surface. This variability can lead to inconsistencies in measurements, which may affect the reliability and repeatability of iPPG results.

Regarding focus on diffuse reflection, iPPG techniques benefit more from capturing diffuse reflection (light that penetrates into the skin and scatters as it interacts with tissues) rather than specular reflection. Diffuse reflection carries information about the blood volume changes beneath the skin surface, which is essential for iPPG.

As illustrated in FIGS. 1A and 1B, filtering out the specular light may reduce a visibility of the skin surface but increase visibility of the diffuse reflection. By minimizing or filtering out specular light, iPPG systems can enhance the quality of the physiological signals extracted and improve the accuracy of heart rate, blood oxygen saturation, and other vital signs monitoring. Thus, separating the specular reflection component from the diffuse reflection component may benefit iPPG techniques. However, as will also be described further below, the specular reflection component may also be used for different analyses.

FIG. 2 illustrates a device 200 of an example implementation of the PPG system described herein. FIG. 2 illustrates an optical PPG module 220, having an illuminator 222 and a detector 224, along with a skin 210 (e.g., a general representation of a patch of a user’s skin). In optical PPG module 220, polarization filtering (e.g., detector 224 having a polarizer) may be implemented to enhance the detection of diffuse reflections 236 from a diffuse 234 in deeper tissue layers of skin 210, and reduce specular reflections 232 from a skin surface of skin 210. This may be achieved by using a polarized light source (e.g., illuminator 222) and a polarization-sensitive detector (e.g., detector 224).

Illuminator 222 may provide linearly or circularly polarized light, which, upon interacting with skin 210, partly transforms into diffuse reflection 236 that carries PPG signal. Detector 224, which may be equipped with a polarizer or polarizer array, may selectively capture this diffuse light or otherwise may provide a decomposed signal (e.g., a polarized light signal for the diffuse reflection component) while minimizing the detection of specularly reflected light (e.g., specular reflection 232), thereby improving the signal quality for more accurate PS-PPG readings. However, in other examples, specular reflection 232 may also be used for different analyses.

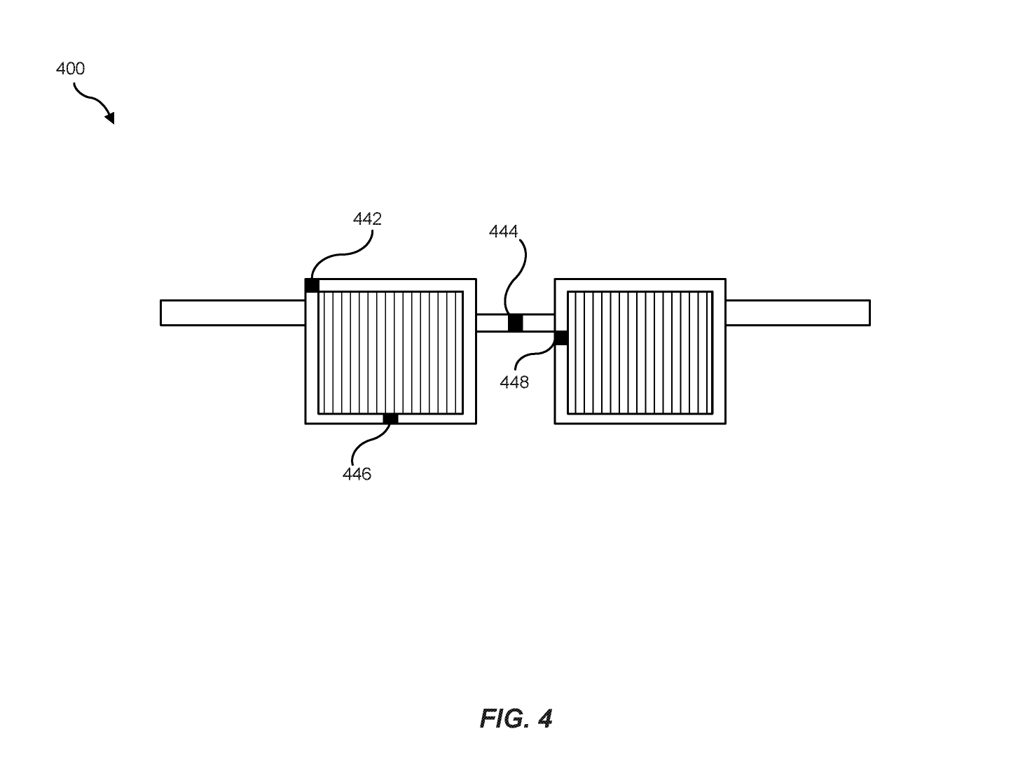

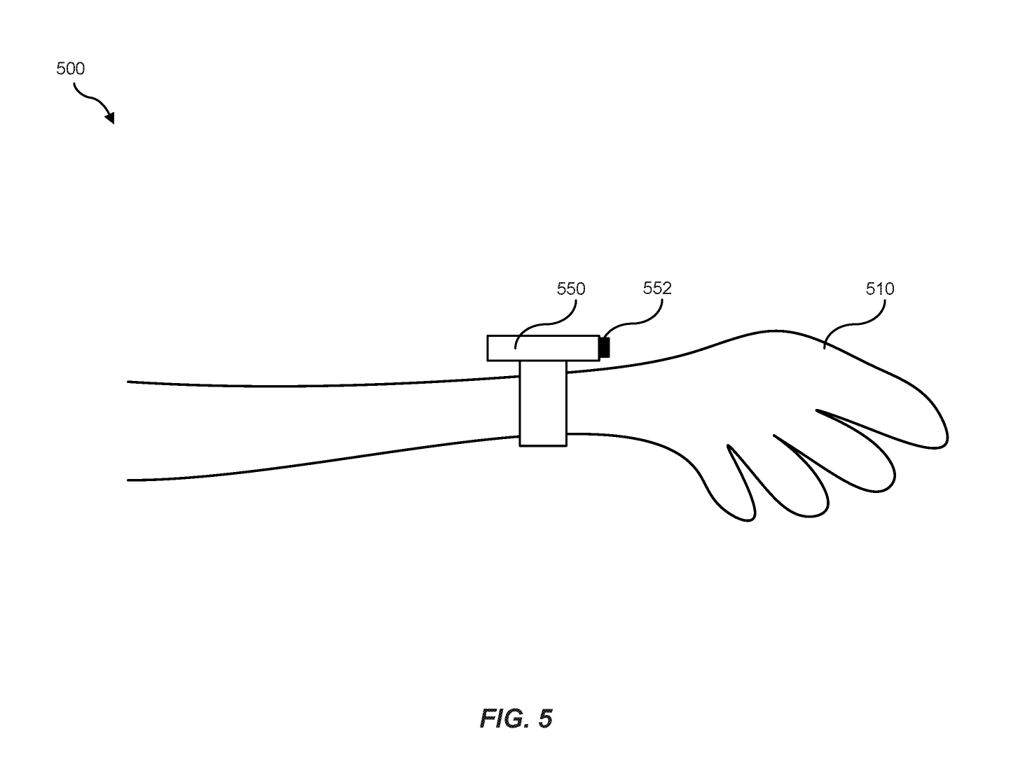

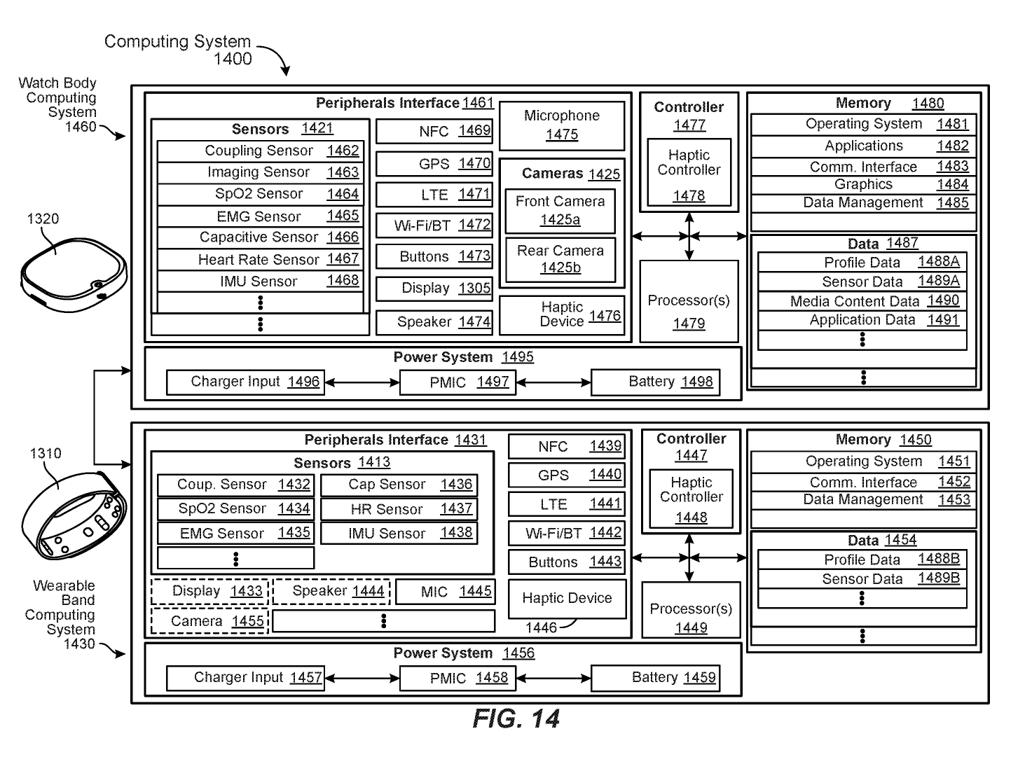

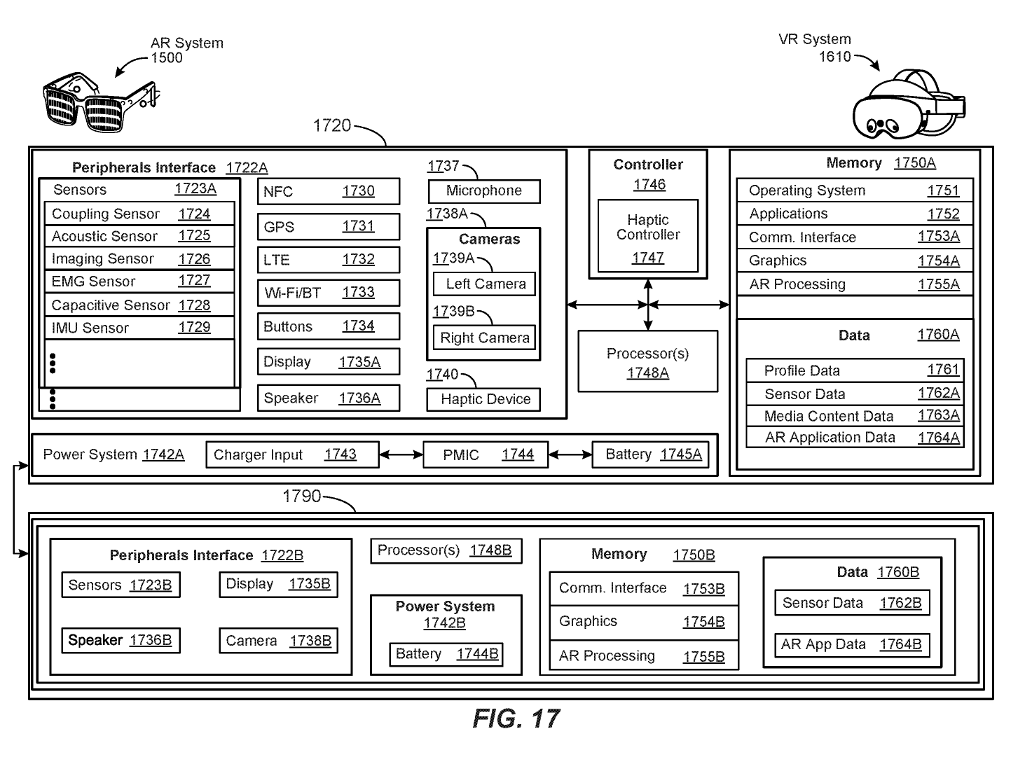

Optical PPG module 220 represents a simplified example of components. In some examples, optical PPG module 220 may include a processor/controller and/or a memory, which in some implementations may be integrated with optical PPG module 220 and/or a component therein (e.g., part of detector 224), and in other implementations may be shared with or otherwise represented by another processor and/or memory within an overall system/device (see, e.g., FIGS. 14 and 17 of example processors and memory). In other words, optical PPG module 220 may be integrated with or otherwise incorporated into another system/device having its components spread out within the device and/or shared by other components such that in some implementations, optical PPG module 220 may not be a discrete packaged component, as will be explained further below with respect to FIGS. 4 and 5.

FIG. 3 illustrates a flowchart 300 of a basic process for iPPG. As illustrated in FIG. 3, at 310 a light source (e.g., illuminator 222) may provide a light directed onto a skin surface 312 (e.g., the surface of skin 210). The light may reflect off of the skin surface, which may include both specular light (e.g., specular reflection 232) and diffuse light (e.g., diffuse reflection 236).

Filtering may occur at reflection type 314 for filtering light based on reflection type. For example, specular light may be filtered out at 330 or otherwise reduced (e.g., using polarization filters), and diffuse light reflection captured at 320. The captured diffuse reflection may be analyzed for blood volume changes at 322, to extract iPPG signals at 324, and further monitor vital signs at 326, as will be described further below.

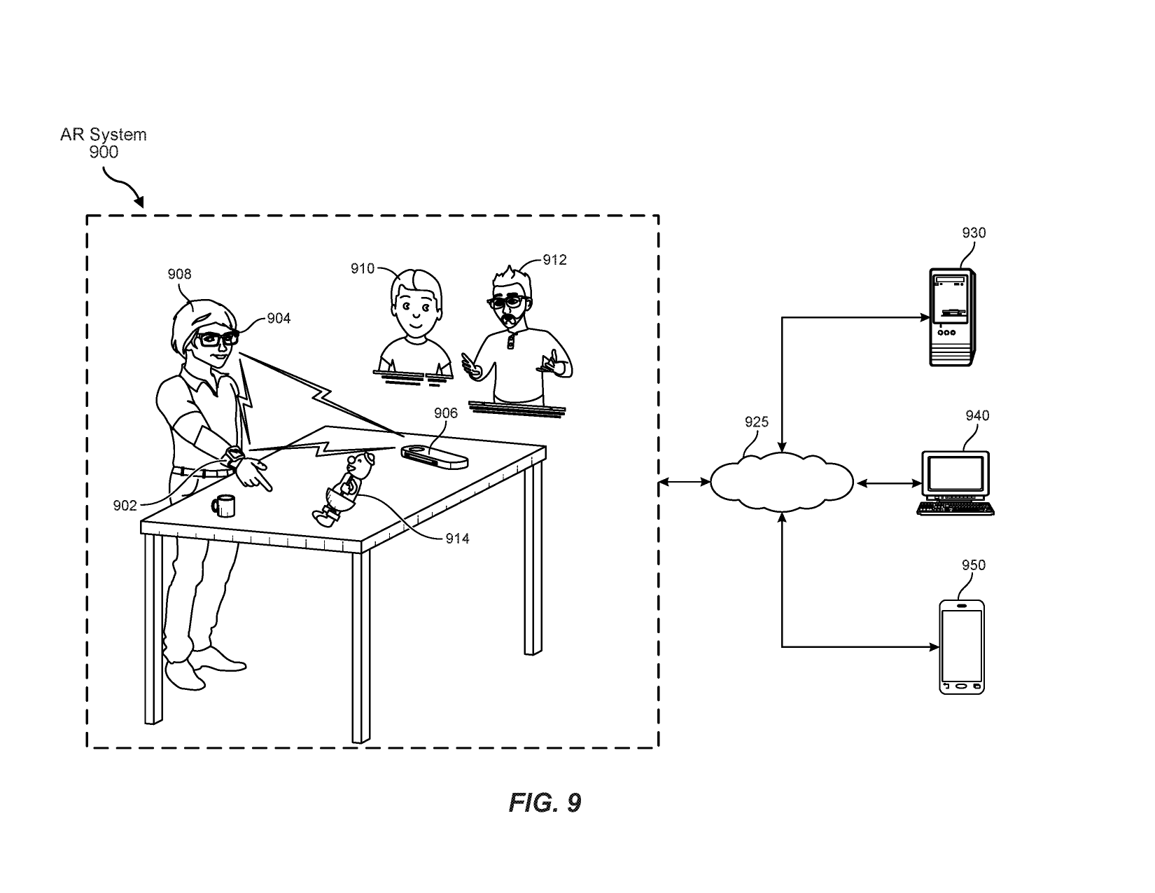

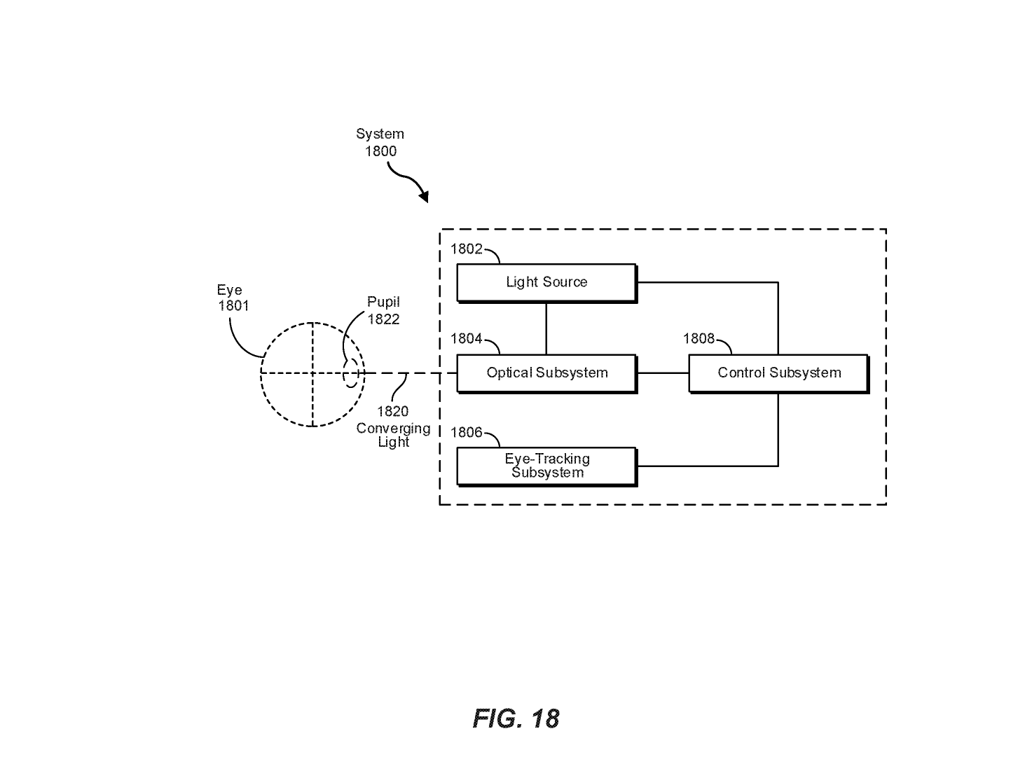

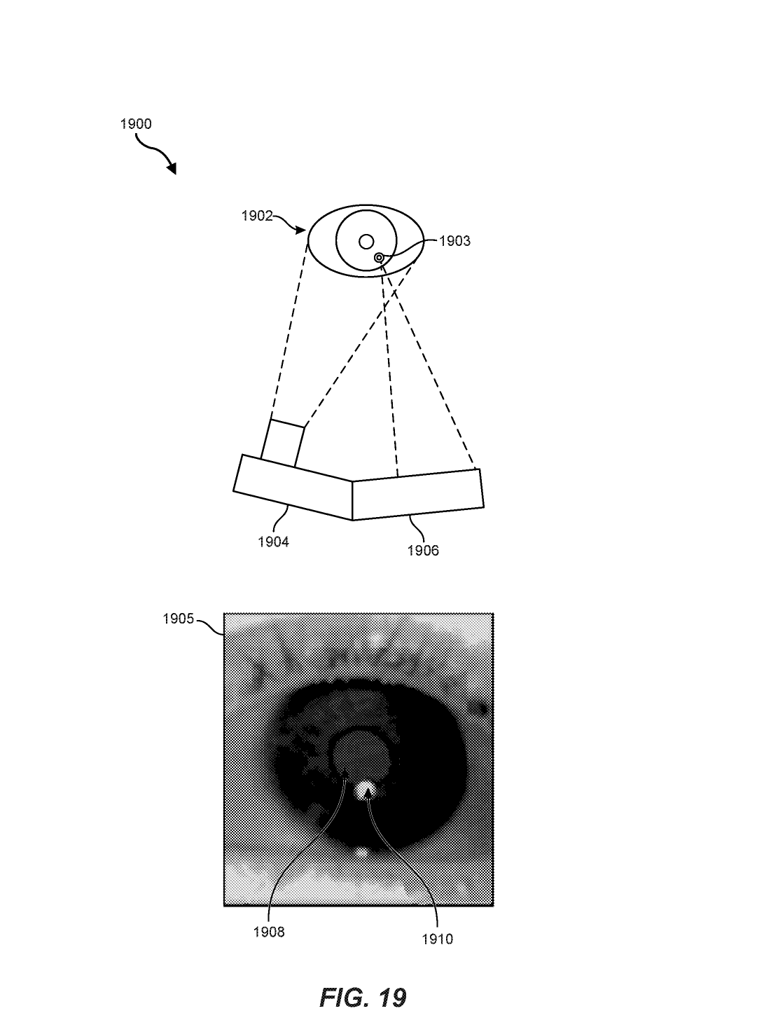

FIG. 4 illustrates a device 400 of another example implementation of the PPG system described herein. Device 400 may correspond to a glasses form factor or other head-mounted display (corresponding to systems described in FIGS. 9, 10, 11, 12A, 12B, 15, 16A, 16B,and 17) having an optical PPG module (e.g., optical PPG module 220). FIG. 4 illustrates various example configurations of optical sensors (corresponding to detector 224), such as a world-facing camera 442 (e.g., oriented away from a user/wearer of the glasses and toward the environment, such as a front-facing camera) which may detect heart rate of others (e.g., not the user), as well as other world- or user-facing cameras that may be used for various other features. For example, a face tracking camera 446 (e.g., a camera for tracking face orientations and/or recognize faces, which may be a world-facing camera), an eye tracking camera 448 (e.g., a camera for tracking the user’s eye gaze/position, corresponding to system described in FIGS. 18 and 19), etc. In other words, the polarized optical sensor may be integrated with or otherwise used with other optical sensors that are used for other features (e.g., the face tracking (FT) camera and/or the eye tracking (ET) camera). In some implementations, a polarized light source (e.g., illuminator 222) may be incorporated near the optical sensor, such as near and/or closely integrated with world-facing camera 442, face tracking camera 446, and/or eye tracking camera 448 such that the polarized light may be oriented in a direction that allows the corresponding optical sensor to capture reflected light (e.g., light reflected from skin). In such examples, optical PPG module 220 may be incorporated as the optical sensor. Alternatively, or in addition, the polarized light source may be located elsewhere on device 400. For instance, the polarized light source may be shared by more than one optical sensor in that the light reflected off the skin from a given polarized light source may be captured by more than one optical sensor. Moreover, in some examples, a given optical sensor may capture light from more than one polarized light source.

In other implementations, device 400 may include a dedicated optical PPG module 444 (corresponding to optical PPG module 220). For instance, optical PPG module 444 may be configured for optical PPG rather than having components shared or otherwise incorporated with other modules, which in some examples may allow more optimal positioning of optical PPG module 444 (e.g., on a bridge of the glasses) for more consistent measurements.

In some examples, device 400 may incorporate additional or fewer optical PPG modules, which may also be located at different various positions around a frame of device 400. For instance, different world- or user-facing implementations of the optical PPG module may allow biometric measurements at different locations, such as different portions of the user’s skin (providing multiple detection points), and/or other people at different positions with respect to device 400 or multiple detection points for the other people. In addition, as described above, a processor/controller and/or memory may be incorporated within the respective optical PPG module and/or may be incorporated with or shared with processors and/or memory of device 400 (such as in the systems in FIG. 17).

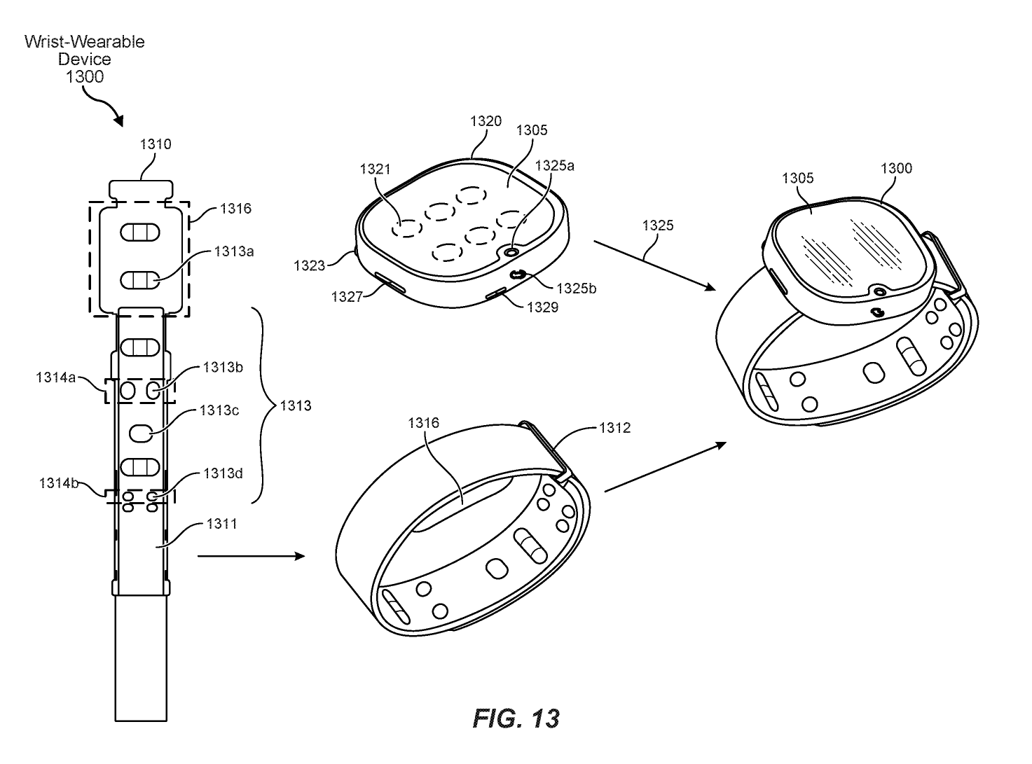

FIG. 5 illustrates a device 500 of another example implementation of the PPG system described herein. Device 500 may correspond to a wrist wearable form factor (e.g., watch, band, etc., corresponding to systems in FIG. 13 and 14) such as a watch 550. FIG. 5 illustrates various example configurations of optical sensors, such as a world-facing camera 552 (e.g., a gesture detection camera) that is adjacent to the skin and accordingly able to capture the user’s skin. World-facing camera 552 may correspond to a camera for capturing gestures performed by a hand 510 of a user/wearer of device 500, for example for interpreting gestures into user inputs. A polarized light source (e.g., illuminator 222) may be near or otherwise closely integrated with world-facing camera 552.

IPPG (e.g., optical PPG module 220) may be implemented on a wrist-based camera to provide either a single detection point (e.g., by itself) or an additional point of measurement (e.g., in addition to detection points from other devices such as device 400 or other head-mounted or wearable device). For instance, biometric measurements may be made using available captured light, which may correspond to one or more detection points. In addition or alternatively, more than one type of biometric measurement (as will be described further below) may be made using different detection points and/or any combinations thereof. In some implementations, the locations of the detection points may correspond to certain types of biometric measurements.

In some examples, device 500 may incorporate additional or fewer optical PPG modules, which may also be located at different various positions around device 500. For instance, different world- or user-facing implementations of the optical PPG module may allow biometric measurements at different locations, such as different portions of the user’s skin, and/or other people at different positions with respect to device 500. In addition, as described above, a processor/controller and/or memory may be incorporated within the respective optical PPG module and/or may be incorporated with or shared with processors and/or memory of device 500 (such as in the systems in FIG. 14).

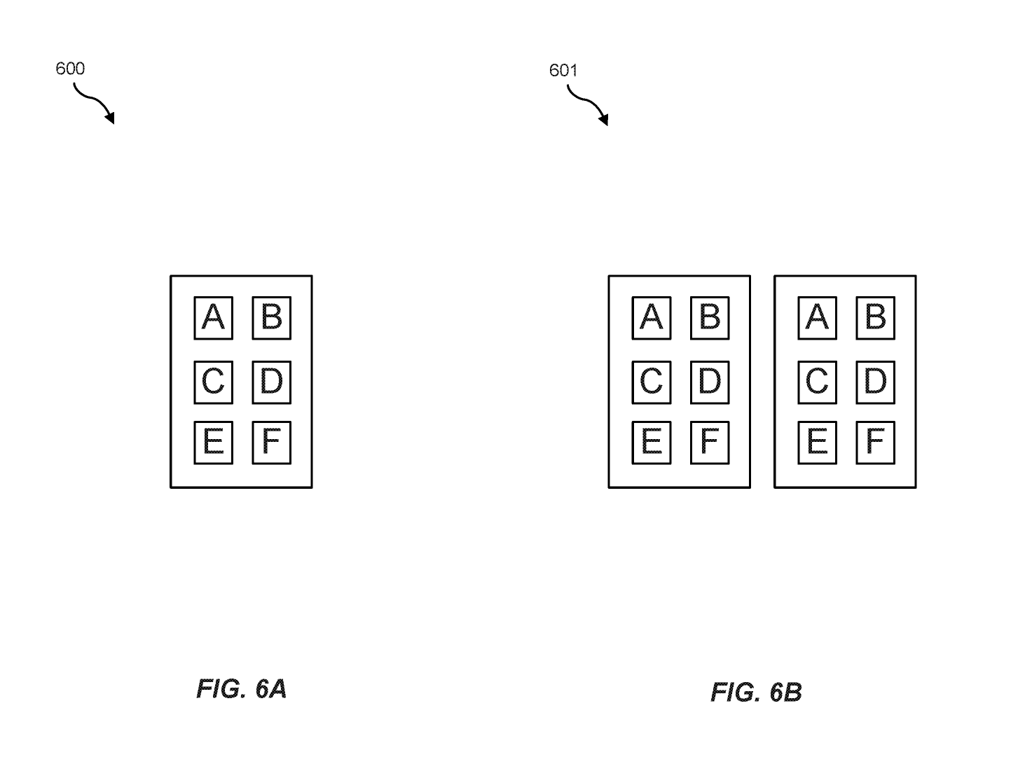

FIGS. 6A-6B illustrate a diagrams of various sensor configurations (e.g., polarizers), including a monochrome configuration 600, and a two-color configuration 601. For polarization-sensitive detection in optical PPG systems, various single-point sensor configurations using polarizers may be designed to meet specific detection needs. Some configurations may use only sensor A, representing a single linear polarizer. This configuration may use one detector with a linear polarizer, which may be straightforward and economical. This may filter light based on a single polarization axis but may not effectively discriminate between specular and diffuse reflections due to its limited polarization differentiation.

Some configurations may use two detectors, A and B, each with a polarizer oriented at 90 degrees to the other. This configuration may enhance the ability to distinguish between different polarization states by measuring light intensity in two perpendicular directions.

Some configurations may use four linear polarizers, A, B, C, and D. With four detectors, each equipped with a polarizer at angles of 0°, 45°, 90°, and 135°, this arrangement may allow for a detailed analysis of the polarization state across multiple orientations.

Some configurations may combine four linear polarizers (A, B, C, D) with two circular polarizers (E, F), one for each direction of circular polarization. This configuration may provide a robust method for distinguishing specular from diffuse reflections across a range of incident angles. Although FIG. 6A illustrates six sensors (A, B, C, D, E, F) for illustrative purposes, it may be understood that configurations having fewer sensors may not include the other sensors.

Dual color detection provides option for detecting blood oxygenation. Dual color detection may include a pair of detector sets, as in FIG. 6B. In addition, each detector may be implemented as a (I) point detector, (II) point detector combined with a scanning system or (III) array of detectors (camera). For example, although FIG. 6B illustrates two detector sets having six sensors, in other implementations each detector set may use a different number of sensors, as described above.

In addition, some sensor configurations may allow separating polarization states (e.g., corresponding to a specular light component and a diffused light component) to allow different analysis on the different polarization states. For instance, a polarized light may be detected and separated by the polarizers into the specular light component and the diffused light component, which may then be separated (e.g., detected and converted into sensor signals for further conversion into data signals for processing) and analyzed. In other instances, the different light components may be selectively captured (e.g., the polarizers and sensors capturing one of the specular light component in one sensing iteration and the diffused light component and on a next sensing iteration).

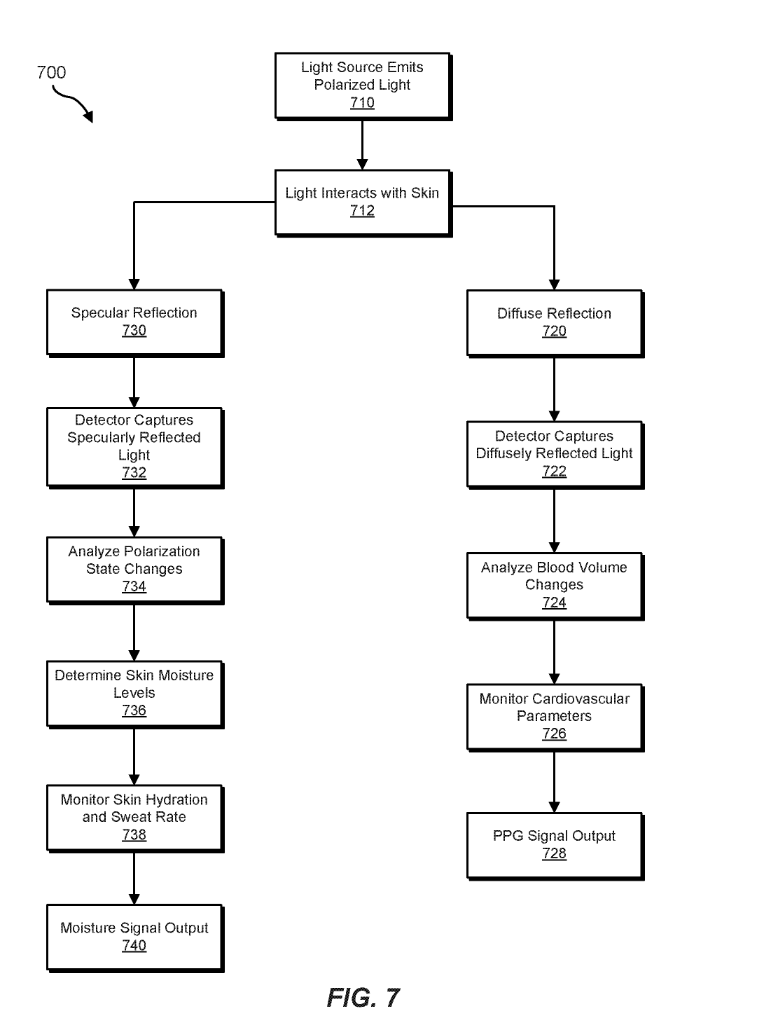

FIG. 7 illustrates a flowchart 700 using both specular and diffuse reflection. FIG. 7 illustrates how polarization-sensitive photoplethysmography (PS-PPG) system as described herein, such as optical PPG module 220 (which may be part of device 400 and/or device 500), utilizes a specialized detector (e.g., detector 224 using sensors described in FIGS. 6A and 6B) capable of analyzing both specular reflections (e.g., specular reflection 232) and diffuse reflections (e.g., diffuse reflection 236) of polarized light (e.g., polarized light 230 from illuminator 222) reflected from a skin (e.g., skin 210). In other words, although the various examples described above referenced filtering out specular reflections, in some implementations, the specular reflections may also be used for biometric measurements (e.g., using the captured specular reflections before filtering and/or recapturing the specular reflections separately from the diffuse reflections).

As illustrated in FIG. 7, a light source (e.g., illuminator 222) may emit polarized light (e.g., polarized light 230) at 710 which may interact with the skin (e.g., skin 210) at 712 to produce specular reflection (e.g., specular reflection 232) at 730 and diffuse reflection (e.g., diffuse reflection 236) at 720. An optical detector (e.g., detector 224 further corresponding to one or more of world-facing camera 442, face tracking camera 446, eye tracking camera 448, optical PPG module 444, and/or world-facing camera 552) of the PPG system may capture the light as reflected off the skin. In some examples, the captured light may correspond to a same patch of skin being captured over time (e.g., using multiple readings/captures to detect changes in the skin over time).

At 730, the polarized light may reflect as specularly reflected light (e.g., specular reflection 232). At 732, the optical detector may capture the specularly reflected light. For example, using one or more polarizer, the optical detector may detect the specularly reflected light and/or otherwise separate a specular light component from the captured light. At 734, the PPG system may analyze polarization state changes (e.g., comparing different specularly reflected light signals as measured over time) to determine skin moisture levels at 736. This further allows the PPG system to monitor skin hydration and sweat rate at 738 to output a moisture signal at 740, which may be used by other systems (e.g., for recalibrating other components such as electrodes or other sensors that require physical contact with skin, as a signal for other analysis/processing, as part of a health related report, etc.).

The light reflected from the skin may also include, at 720, a diffuse reflection (e.g., diffuse reflection 236). The same or another optical detector of the PPG system may capture the diffusely reflected light at 722. For instance, the optical detector may use polarizers to detect the diffusely reflected light and/or otherwise separate a diffuse light component from the captured light, which may happen in parallel with 732 (e.g., same sensor reading) and/or on a separate sensor reading. Using the diffusely reflected light, the PPG system may analyze blood volume changes (e.g., comparing different diffusely reflected light signals as measured over time) at 724 to further monitor cardiovascular parameters at 726. At 728, the PPG system may output a corresponding PPG signal based on the monitoring, which may be used by other systems (e.g., as an input for additional analysis/processing, as part of a health related report, etc.).

This dual-capability system is designed to monitor cardiovascular parameters through diffuse reflections, which are indicative of blood volume changes, and to assess skin moisture levels through specular reflections, which are sensitive to changes in skin surface properties such as moisture due to sweating. By emitting polarized light onto the skin and capturing the light that is reflected back, the system can differentiate between the light components based on their polarization states and reflection types, enabling it to simultaneously monitor heart rate, and other cardiovascular metrics, as well as hydration levels and sweat rate.

In some examples, the photoplethysmography (PPG) system described herein may correspond to a wearable device form factor and may utilize a skin-facing camera (e.g., face tracking camera, eye tracking camera, gesture tracking camera, etc.) to capture subtle variations in light absorption by the skin, which may correspond to blood volume changes in the microvascular bed of tissue. The system provided herein may measure the remote PPG (iPPG) signal by analyzing the intensity fluctuations of reflected light, which is often in the green spectrum, as blood pluses through the capillaries. The iPPG signal may be processed to detect changes in amplitude relative to a baseline, which may be indicative of vasodilation and vasoconstriction events. Vasodilation, which may be characterized by an increase in blood vessel diameter, may result in higher amplitude signals. Vasoconstriction, which may be characterized by a decrease in blood vessel diameter, may result in lower amplitude signals.

By quantifying such amplitude changes, the system may derive various health parameters. For instance, the system may assess vascular compliance and elasticity, which are often critical indicators of cardiovascular health. Additionally, the system may monitor stress levels by evaluating the autonomic nervous system’s influence on peripheral blood flow.

In some examples, the system may track health parameters such as hydration status, as dehydration may lead to reduced blood volume and altered vascular responses. The system may also evaluate endothelial function, which may be crucial for understanding the risk of atherosclerosis and other vascular diseases. The system described herein advantageously provides non-invasive, continuous monitoring capability for personalized health management and early detection of cardiovascular anomalies.

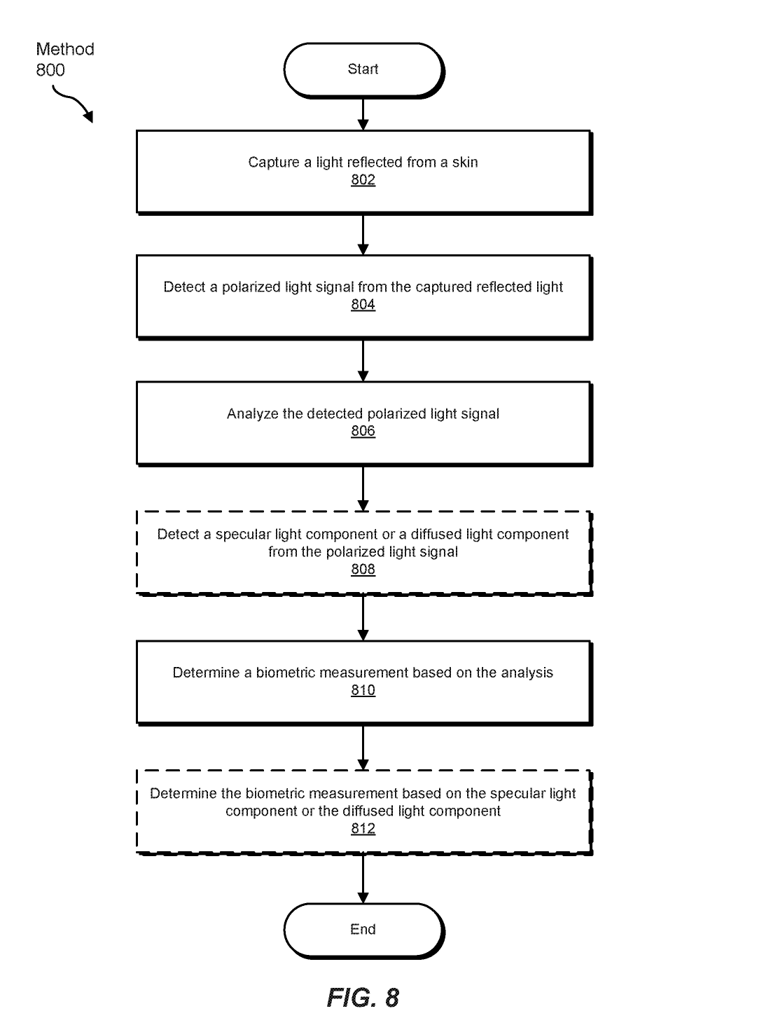

Turning now to FIG. 8, FIG. 8 is a flow diagram of an example computer-implemented method 800 for polarization sensitive biometric monitoring. The steps shown in FIG. 8 may be performed by any suitable computer-executable code and/or computing system, including the system(s) illustrated in FIGS. 2, 4, 5, and/or 6. In one example, each of the steps shown in FIG. 8 may represent an algorithm whose structure includes and/or is represented by multiple sub-steps, examples of which will be provided in greater detail below.

As illustrated in FIG. 8 at step 802 one or more of the systems described herein capture a light reflected from a skin. For example, an optical sensor (e.g., detector 224 further corresponding to one or more of world-facing camera 442, face tracking camera 446, eye tracking camera 448, optical PPG module 444, and/or world-facing camera 552) may capture light (e.g., specular reflection 232 and/or diffuse reflection 236) reflected off skin (e.g., skin 210). In some examples, the light may be from a polarized light source (e.g., illuminator 222), which may provide linearly polarized light and/or circularly polarized light. Further, in some examples, the optical sensor may be configured to measure a same patch of skin over time (e.g., by identifying/detecting a particular patch of skin which may be smaller than a total field of view of the camera).

At step 804 one or more of the systems described herein may detect a polarized light signal from the captured reflected light. For example, the optical sensor may include one or more of a linear polarizer (e.g., a 0-degree polarizer, a 45-degree polarizer, a 90-degree polarizer, and/or a 135-degree polarizer), an orthogonal polarizer, and/or a circular polarizer, and may also be a point detector, a scanning system, and/or an array of detectors, such as one or more of the examples described in connection with FIGS. 6A and 6B. In some examples, the optical sensor is configured for dual color detection. Further, in some examples, the optical sensor may also detect specular light, separately and/or with diffused light. In addition, the optical sensor may detect the light reflected off the same patch of skin over time.

At step 806 one or more of the systems described herein may analyze the detected polarized light signal. For example, the analysis may include detecting changes over time for the detected light reflected off the same patch of skin. In some examples, the analysis may incorporate, optionally at step 808, detecting a specular light component (e.g., specular reflection 232) and/or a diffused light component (e.g., diffuse reflection 236) from the polarized light signal. As detailed above, the different light components may be separated (e.g., by the polarizers) in a given light measurement, and/or may be measured separately (e.g., separate light measurements), which may be repeated in any combination over time for multiple measurements.

At step 810 one or more of the systems described herein may determine a biometric measurement based on the analysis. The systems described herein may perform step 810 in a variety of ways, which may optionally include step 812 of determining the biometric measurement based on the specular light component and/or the diffused light component. In some examples, the biometric measurement may be based on analyzing the diffuse light over time to determine blood volume changes, monitor cardiovascular parameters, perform photoplethysmography (PPG), determine heart rate measurement, take blood oxygen measurements, and/or take breathing rate measurements. Further, when the optical sensor is configured to capture specular light from the skin, the biometric measurement may be based on analyzing the specular light over time to analyze polarization state changes, determine skin moisture levels, monitor skin hydration, monitor sweat rate, and/or detect an oiliness of the skin.

Accordingly, the biometric measurement may represent or otherwise include one or more different measurements, such as measurements based on the specular light component (e.g., moisture-related skin measurements) and/or measurements based on the diffused light component (e.g., cardiovascular-related measurements).

As detailed above, photoplethysmography (IPPG) is a widely used technology in both clinical and consumer health devices for monitoring heart rate and other cardiovascular parameters. The technology operates by emitting light into the skin and measuring the amount of light either transmitted or reflected back from the blood vessels, which varies with each heartbeat due to changes in blood volume. Reflective sensors are common in wearable devices like smartwatches and fitness bands. These sensors are placed directly on the skin and measure the light reflected back from the blood vessels. Transmissive sensors are often used in devices like pulse oximeters, where the sensor is placed on a thin part of the patient’s body, such as a fingertip or earlobe, and measures the light passing through. Significant challenges may arise from motion artifacts caused by user movements, which may distort the readings. Variability in ambient light may also affect the sensor’s ability to accurately detect blood volume changes. Differences in skin tone, thickness, and presence of tattoos may impact the accuracy of heart rate readings. In addition, reflections from oily or wet skin, or from external sources, may significantly degrade the quality of the signal by affecting the signal-to-noise ratio (SNR). To improve accuracy, especially in calculating blood oxygen levels, sensors using multiple wavelengths of light have been developed. Algorithms that adapt to different skin types and conditions, as well as to motion and ambient light variations, may be integrated to enhance measurement accuracy. Combining data from accelerometers and/or other sensors may help to filter out motion artifacts and improve the reliability of heart rate measurements.

Example 1: A device comprising: an optical sensor comprising a polarizer, wherein the optical sensor is configured to: capture a polarized light from a skin; detect a diffused light component from the captured polarized light using the polarizer; and perform a biometric measurement based on the detected diffused light component.

Example 2: The device of Example 1, wherein the biometric measurement corresponds to at least one of: analyzing blood volume changes; monitoring cardiovascular parameters; photoplethysmography (PPG); heart rate measurement; blood oxygen measurement; or breathing rate measurement.

Example 3: The device of Example 1 or 2, further comprising a polarized light source configured to provide linearly polarized light or circularly polarized light to the skin.

Example 4: The device of any of Examples 1-3, wherein the polarizer comprises at least one of: a linear polarizer; an orthogonal polarizer; a circular polarizer; or a plurality of linear polarizers.

Example 5: The device of Example 4, wherein the plurality of linear polarizers includes a 0-degree polarizer, a 45-degree polarizer, a 90-degree polarizer, and a 135-degree polarizer.

Example 6: The device of any of Examples 1-5, wherein the optical sensor is further configured to: capture a specular light reflected from the skin; and perform the biometric measurement based on the captured specular light.

Example 7: The device of Example 6, wherein the biometric measurement based on the captured specular light corresponds to at least one of: analyzing polarization state changes; determining skin moisture levels; monitoring skin hydration; monitoring sweat rate; or detecting an oiliness of the skin.

Example 8: The device of any of Examples 1-7, wherein the optical sensor is configured for dual color detection.

Example 9: The device of any of Examples 1-8, wherein the optical sensor comprises at least one of: a point detector; a scanning system; or an array of detectors.

Example 10: The device of any of Examples 1-9, wherein the device comprises a smart glasses form factor and the optical sensor comprises at least one of: an eye tracking camera; or a face tracking camera.

Example 11: The device of any of Examples 1-10, wherein the device comprises a wrist wearable device form factor and the optical sensor comprises a camera for gesture detection.

Example 12: The device of any of Examples 1-11, wherein the optical sensor is configured to measure a same patch of the skin over time.

Example 13: The device of any of Examples 1-12, wherein the optical sensor is configured to analyze an intensity of a fluctuation of the diffused light with respect to a baseline.

Example 14: The device of Example 13, wherein a high amplitude relative to the baseline indicates an increase in a blood vessel diameter and a low amplitude relative to the baseline indicates a decrease in the blood vessel diameter.

Example 15: A system comprising: a polarized light source configured to provide linearly polarized light or circularly polarized light to a skin; and an optical sensor comprising a polarizer, wherein the optical sensor is configured to: capture a polarized light from the skin; detect a diffused light component from the captured polarized light using the polarizer; and perform a biometric measurement based on the detected diffused light component.

Example 16: The system of Example 15, wherein the biometric measurement corresponds to at least one of: analyzing blood volume changes; monitoring cardiovascular parameters; photoplethysmography (PPG); heart rate measurement; blood oxygen measurement; or breathing rate measurement.

Example 17: The system of Example 15 or 16, wherein: the optical sensor is further configured to: detect a specular light component from the captured polarized light using the polarizer; and perform the biometric measurement based on the detected specular light component; and the biometric measurement based on the detected specular light component corresponds to at least one of: analyzing polarization state changes; determining skin moisture levels; monitoring skin hydration; monitoring sweat rate; or detecting an oiliness of the skin.

Example 18: The system of any of Examples 15-17, wherein: the polarizer comprises at least one of: a linear polarizer; an orthogonal polarizer; a circular polarizer; or a plurality of linear polarizers that includes a 0-degree polarizer, a 45-degree polarizer, a 90-degree polarizer, and a 135-degree polarizer; and the optical sensor comprises at least one of: a dual color detector; a point detector; a scanning system; or an array of detectors.

Example 19: A method comprising: capturing a light reflected from a skin; detecting a polarized light signal from the captured reflected light; analyzing the detected polarized light signal; and determining a biometric measurement based on the analysis.

Example 20: The method of Example 19, further comprising: detecting a specular light component or a diffused light component from the polarized light signal; and determining the biometric measurement based on the specular light component or the diffused light component.

Example 21: A device comprising: an optical sensor including a polarizer and configured to take a biometric measurement based on capturing diffused light from skin over time.

Example 22: The device of Example 21, wherein the optical sensor comprises at least one of: a linear polarizer; an orthogonal polarizer; or a circular polarizer.

Example 23: The device of Example 21 or 22, wherein the optical sensor comprises a plurality of linear polarizers.

Example 24: The device of Example 23, wherein the plurality of linear polarizers includes a 0-degree polarizer, a 45-degree polarizer, a 90-degree polarizer, and a 135-degree polarizer.

Example 25: The device of any of Examples 21-24, wherein the optical sensor is configured for dual color detection.

Example 26: The device of any of Examples 21-25, wherein the optical sensor comprises at least one of: a point detector; a scanning system; or an array of detectors.

Example 27: The device of any of Examples 21-26, further comprising a polarized light source.

Example 28: The device of Example 27, wherein the polarized light source provides linearly polarized light or circularly polarized light.

Example 29: The device of any of Examples 21-28, wherein the optical sensor is configured to perform at least on of: analyzing blood volume changes; monitoring cardiovascular parameters; photoplethysmography (PPG); heart rate measurement; blood oxygen measurement; or breathing rate measurement.

Example 30: The device of any of Examples 21-29, wherein the optical sensor is configured to capture specular light from the skin.

Example 31: The device of Example 30, wherein the optical sensor is configured to perform at least one of: analyzing polarization state changes; determining skin moisture levels; monitoring skin hydration; monitoring sweat rate; or detecting an oiliness of the skin.

Example 32: The device of any of Examples 21-31, wherein the device comprises a smart glasses form factor.

Example 33: The device of any of Examples 21-32, wherein the optical sensor comprises at least one of: an eye tracking camera; or a face tracking camera.

Example 34: The device of any of Examples 21-33, wherein the device comprises a wrist wearable device form factor.

Example 35: The device of Example 34, wherein the optical sensor comprises a camera for gesture detection.

Example 36: The device of any of Examples 21-35, wherein the optical sensor is configured to measure a same patch of skin over time.

Example 37: The device of any of Examples 21-36, wherein the optical sensor is configured to analyze an intensity of a fluctuation of the diffused light with respect to a baseline.

Example 38: The device of Example 37, wherein a high amplitude relative to the baseline indicates an increase in blood vessel diameter and a low amplitude relative to the baseline indicates a decrease in blood vessel diameter.

Embodiments of the present disclosure may include or be implemented in conjunction with various types of Artificial-Reality (AR) systems. AR may be any superimposed functionality and/or sensory-detectable content presented by an artificial-reality system within a user’s physical surroundings. In other words, AR is a form of reality that has been adjusted in some manner before presentation to a user. AR can include and/or represent virtual reality (VR), augmented reality, mixed AR (MAR), or some combination and/or variation of these types of realities. Similarly, AR environments may include VR environments (including non-immersive, semi-immersive, and fully immersive VR environments), augmented-reality environments (including marker-based augmented-reality environments, markerless augmented-reality environments, location-based augmented-reality environments, and projection-based augmented-reality environments), hybrid-reality environments, and/or any other type or form of mixed- or alternative-reality environments.

AR content may include completely computer-generated content or computer-generated content combined with captured (e.g., real-world) content. Such AR content may include video, audio, haptic feedback, or some combination thereof, any of which may be presented in a single channel or in multiple channels (such as stereo video that produces a three-dimensional (3D) effect to the viewer). Additionally, in some embodiments, AR may also be associated with applications, products, accessories, services, or some combination thereof, that are used to, for example, create content in an artificial reality and/or are otherwise used in (e.g., to perform activities in) an artificial reality.

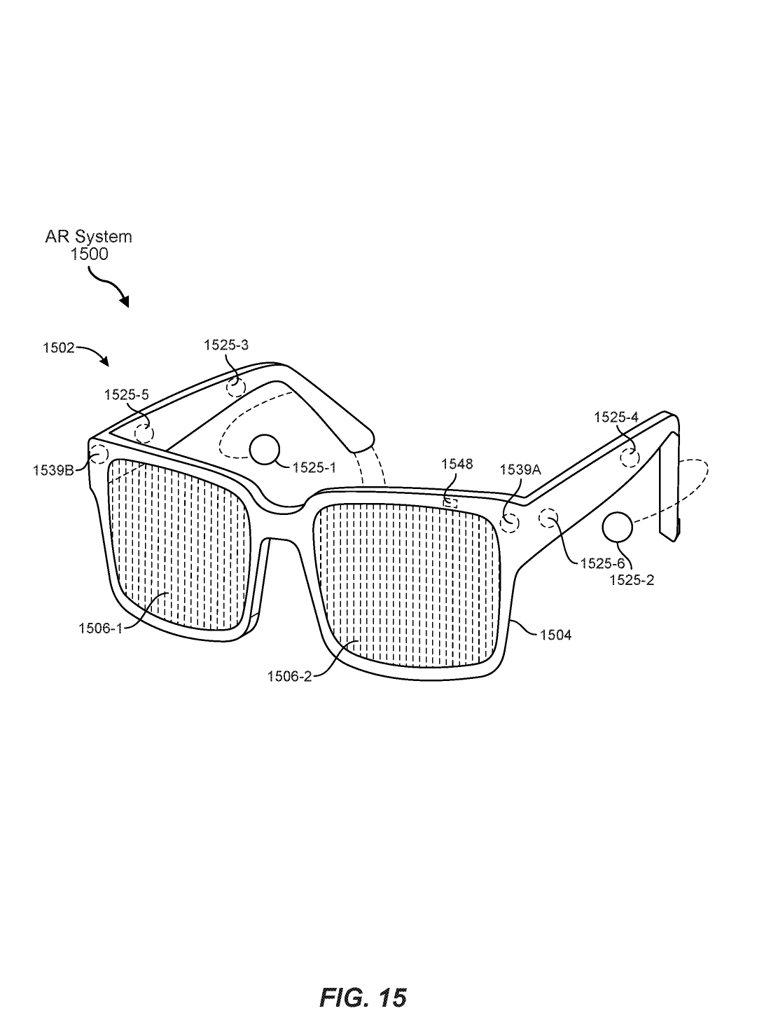

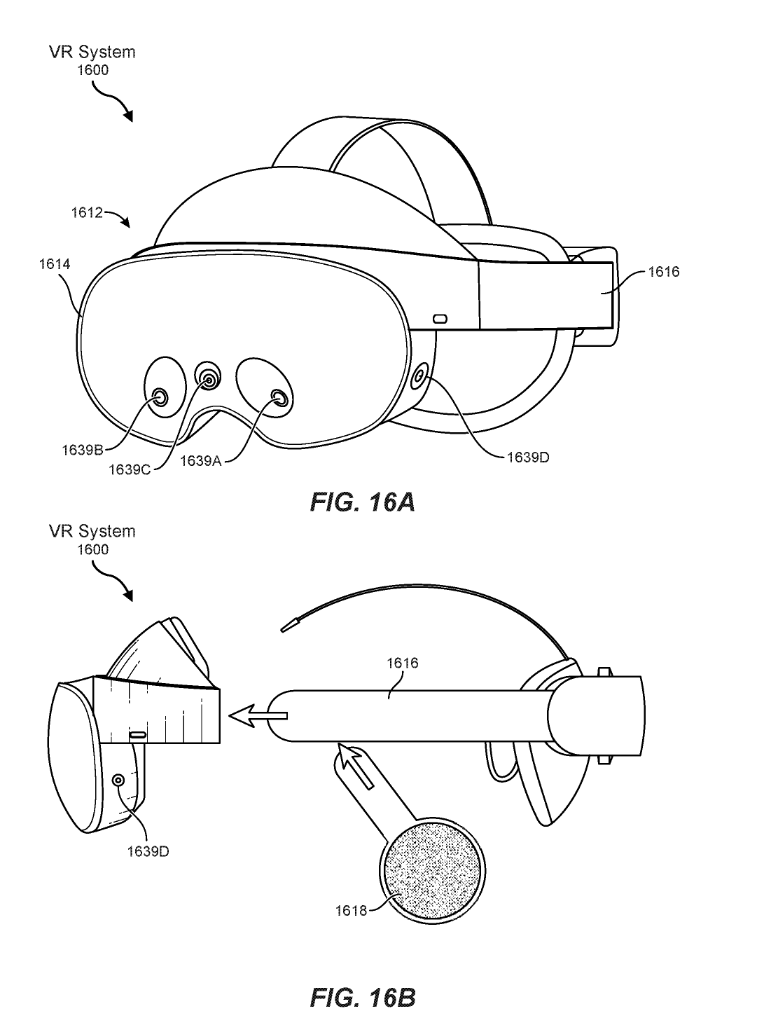

AR systems may be implemented in a variety of different form factors and configurations. Some AR systems may be designed to work without near-eye displays (NEDs). Other AR systems may include a NED that also provides visibility into the real world (such as, e.g., augmented-reality system 1500 in FIG. 15) or that visually immerses a user in an artificial reality (such as, e.g., virtual-reality system 1600 in FIGS. 16A and 16B). While some AR devices may be self-contained systems, other AR devices may communicate and/or coordinate with external devices to provide an AR experience to a user. Examples of such external devices include handheld controllers, mobile devices, desktop computers, devices worn by a user, devices worn by one or more other users, and/or any other suitable external system.

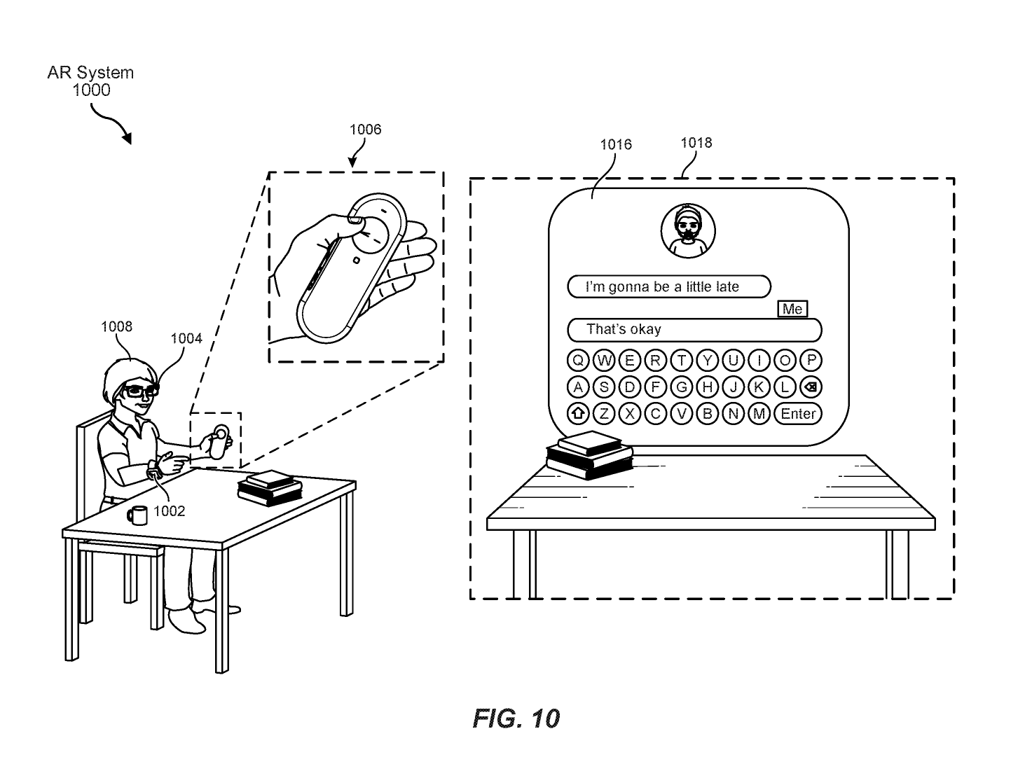

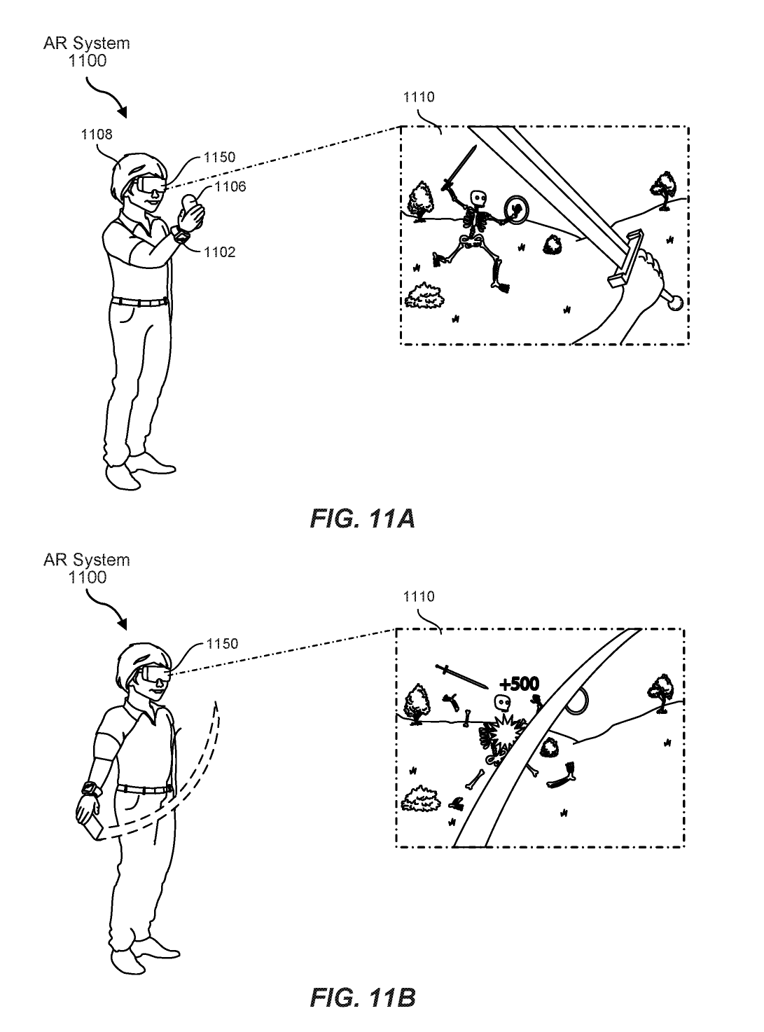

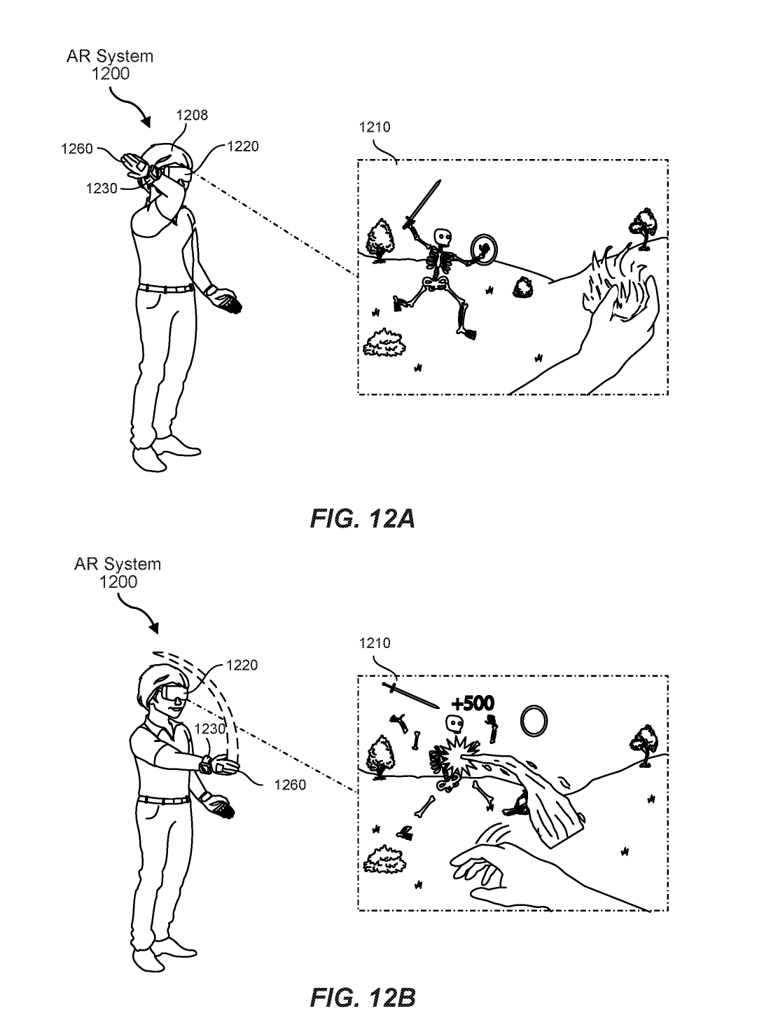

FIGS. 9-12B illustrate example artificial-reality (AR) systems in accordance with some embodiments. FIG. 9 shows a first AR system 900 and first example user interactions using a wrist-wearable device 902, a head-wearable device (e.g., AR glasses 1500), and/or a handheld intermediary processing device (HIPD) 906. FIG. 10 shows a second AR system 1000 and second example user interactions using a wrist-wearable device 1002, AR glasses 1004, and/or an HIPD 1006. FIGS. 11A and 11B show a third AR system 1100 and third example user 1108 interactions using a wrist-wearable device 1102, a head-wearable device (e.g., VR headset 1150), and/or an HIPD 1106. FIGS. 12A and 12B show a fourth AR system 1200 and fourth example user 1208 interactions using a wrist-wearable device 1230, VR headset 1220, and/or a haptic device 1260 (e.g., wearable gloves).

A wrist-wearable device 1300, which can be used for wrist-wearable device 902, 1002, 1102, 1230, and one or more of its components, are described below in reference to FIGS. 13 and 14; head-wearable devices 1500 and 1600, which can respectively be used for AR glasses 904, 1004 or VR headset 1150, 1220, and their one or more components are described below in reference to FIGS. 15-17.

Referring to FIG. 9, wrist-wearable device 902, AR glasses 904, and/or HIPD 906 can communicatively couple via a network 925 (e.g., cellular, near field, Wi-Fi, personal area network, wireless LAN, etc.). Additionally, wrist-wearable device 902, AR glasses 904, and/or HIPD 906 can also communicatively couple with one or more servers 930, computers 940 (e.g., laptops, computers, etc.), mobile devices 950 (e.g., smartphones, tablets, etc.), and/or other electronic devices via network 925 (e.g., cellular, near field, Wi-Fi, personal area network, wireless LAN, etc.).

In FIG. 9, a user 908 is shown wearing wrist-wearable device 902 and AR glasses 904 and having HIPD 906 on their desk. The wrist-wearable device 902, AR glasses 904, and HIPD 906 facilitate user interaction with an AR environment. In particular, as shown by first AR system 900, wrist-wearable device 902, AR glasses 904, and/or HIPD 906 cause presentation of one or more avatars 910, digital representations of contacts 912, and virtual objects 914. As discussed below, user 908 can interact with one or more avatars 910, digital representations of contacts 912, and virtual objects 914 via wrist-wearable device 902, AR glasses 904, and/or HIPD 906.

User 908 can use any of wrist-wearable device 902, AR glasses 904, and/or HIPD 906 to provide user inputs. For example, user 908 can perform one or more hand gestures that are detected by wrist-wearable device 902 (e.g., using one or more EMG sensors and/or IMUs, described below in reference to FIGS. 13 and 14) and/or AR glasses 904 (e.g., using one or more image sensor or camera, described below in reference to FIGS. 15-10) to provide a user input. Alternatively, or additionally, user 908 can provide a user input via one or more touch surfaces of wrist-wearable device 902, AR glasses 904, HIPD 906, and/or voice commands captured by a microphone of wrist-wearable device 902, AR glasses 904, and/or HIPD 906. In some embodiments, wrist-wearable device 902, AR glasses 904, and/or HIPD 906 include a digital assistant to help user 908 in providing a user input (e.g., completing a sequence of operations, suggesting different operations or commands, providing reminders, confirming a command, etc.). In some embodiments, user 908 can provide a user input via one or more facial gestures and/or facial expressions. For example, cameras of wrist-wearable device 902, AR glasses 904, and/or HIPD 906 can track eyes of user 908 for navigating a user interface.

Wrist-wearable device 902, AR glasses 904, and/or HIPD 906 can operate alone or in conjunction to allow user 908 to interact with the AR environment. In some embodiments, HIPD 906 is configured to operate as a central hub or control center for the wrist-wearable device 902, AR glasses 904, and/or another communicatively coupled device. For example, user 908 can provide an input to interact with the AR environment at any of wrist-wearable device 902, AR glasses 904, and/or HIPD 906, and HIPD 906 can identify one or more back-end and front-end tasks to cause the performance of the requested interaction and distribute instructions to cause the performance of the one or more back-end and front-end tasks at wrist-wearable device 902, AR glasses 904, and/or HIPD 906. In some embodiments, a back-end task is a background processing task that is not perceptible by the user (e.g., rendering content, decompression, compression, etc.), and a front-end task is a user-facing task that is perceptible to the user (e.g., presenting information to the user, providing feedback to the user, etc.). HIPD 906 can perform the back-end tasks and provide wrist-wearable device 902 and/or AR glasses 904 operational data corresponding to the performed back-end tasks such that wrist-wearable device 902 and/or AR glasses 904 can perform the front-end tasks. In this way, HIPD 906, which has more computational resources and greater thermal headroom than wrist-wearable device 902 and/or AR glasses 904, performs computationally intensive tasks and reduces the computer resource utilization and/or power usage of wrist-wearable device 902 and/or AR glasses 904.

In the example shown by first AR system 900, HIPD 906 identifies one or more back-end tasks and front-end tasks associated with a user request to initiate an AR video call with one or more other users (represented by avatar 910 and the digital representation of contact 912) and distributes instructions to cause the performance of the one or more back-end tasks and front-end tasks. In particular, HIPD 906 performs back-end tasks for processing and/or rendering image data (and other data) associated with the AR video call and provides operational data associated with the performed back-end tasks to AR glasses 904 such that the AR glasses 904 perform front-end tasks for presenting the AR video call (e.g., presenting avatar 910 and digital representation of contact 912).

In some embodiments, HIPD 906 can operate as a focal or anchor point for causing the presentation of information. This allows user 908 to be generally aware of where information is presented. For example, as shown in first AR system 900, avatar 910 and the digital representation of contact 912 are presented above HIPD 906. In particular, HIPD 906 and AR glasses 904 operate in conjunction to determine a location for presenting avatar 910 and the digital representation of contact 912. In some embodiments, information can be presented a predetermined distance from HIPD 906 (e.g., within 5 meters). For example, as shown in first AR system 900, virtual object 914 is presented on the desk some distance from HIPD 906. Similar to the above example, HIPD 906 and AR glasses 904 can operate in conjunction to determine a location for presenting virtual object 914. Alternatively, in some embodiments, presentation of information is not bound by HIPD 906. More specifically, avatar 910, digital representation of contact 912, and virtual object 914 do not have to be presented within a predetermined distance of HIPD 906.

User inputs provided at wrist-wearable device 902, AR glasses 904, and/or HIPD 906 are coordinated such that the user can use any device to initiate, continue, and/or complete an operation. For example, user 908 can provide a user input to AR glasses 904 to cause AR glasses 904 to present virtual object 914 and, while virtual object 914 is presented by AR glasses 904, user 908 can provide one or more hand gestures via wrist-wearable device 902 to interact and/or manipulate virtual object 914.

FIG. 10 shows a user 1008 wearing a wrist-wearable device 1002 and AR glasses 1004, and holding an HIPD 1006. In second AR system 1000, the wrist-wearable device 1002, AR glasses 1004, and/or HIPD 1006 are used to receive and/or provide one or more messages to a contact of user 1008. In particular, wrist-wearable device 1002, AR glasses 1004, and/or HIPD 1006 detect and coordinate one or more user inputs to initiate a messaging application and prepare a response to a received message via the messaging application.

In some embodiments, user 1008 initiates, via a user input, an application on wrist-wearable device 1002, AR glasses 1004, and/or HIPD 1006 that causes the application to initiate on at least one device. For example, in second AR system 1000, user 1008 performs a hand gesture associated with a command for initiating a messaging application (represented by messaging user interface 1016), wrist-wearable device 1002 detects the hand gesture and, based on a determination that user 1008 is wearing AR glasses 1004, causes AR glasses 1004 to present a messaging user interface 1016 of the messaging application. AR glasses 1004 can present messaging user interface 1016 to user 1008 via its display (e.g., as shown by a field of view 1018 of user 1008). In some embodiments, the application is initiated and executed on the device (e.g., wrist-wearable device 1002, AR glasses 1004, and/or HIPD 1006) that detects the user input to initiate the application, and the device provides another device operational data to cause the presentation of the messaging application. For example, wrist-wearable device 1002 can detect the user input to initiate a messaging application, initiate and run the messaging application, and provide operational data to AR glasses 1004 and/or HIPD 1006 to cause presentation of the messaging application. Alternatively, the application can be initiated and executed at a device other than the device that detected the user input. For example, wrist-wearable device 1002 can detect the hand gesture associated with initiating the messaging application and cause HIPD 1006 to run the messaging application and coordinate the presentation of the messaging application.

Further, user 1008 can provide a user input provided at wrist-wearable device 1002, AR glasses 1004, and/or HIPD 1006 to continue and/or complete an operation initiated at another device. For example, after initiating the messaging application via wrist-wearable device 1002 and while AR glasses 1004 present messaging user interface 1016, user 1008 can provide an input at HIPD 1006 to prepare a response (e.g., shown by the swipe gesture performed on HIPD 1006). Gestures performed by user 1008 on HIPD 1006 can be provided and/or displayed on another device. For example, a swipe gestured performed on HIPD 1006 is displayed on a virtual keyboard of messaging user interface 1016 displayed by AR glasses 1004.

In some embodiments, wrist-wearable device 1002, AR glasses 1004, HIPD 1006, and/or any other communicatively coupled device can present one or more notifications to user 1008. The notification can be an indication of a new message, an incoming call, an application update, a status update, etc. User 1008 can select the notification via wrist-wearable device 1002, AR glasses 1004, and/or HIPD 1006 and can cause presentation of an application or operation associated with the notification on at least one device. For example, user 1008 can receive a notification that a message was received at wrist-wearable device 1002, AR glasses 1004, HIPD 1006, and/or any other communicatively coupled device and can then provide a user input at wrist-wearable device 1002, AR glasses 1004, and/or HIPD 1006 to review the notification, and the device detecting the user input can cause an application associated with the notification to be initiated and/or presented at wrist-wearable device 1002, AR glasses 1004, and/or HIPD 1006.

While the above example describes coordinated inputs used to interact with a messaging application, user inputs can be coordinated to interact with any number of applications including, but not limited to, gaming applications, social media applications, camera applications, web-based applications, financial applications, etc. For example, AR glasses 1004 can present to user 1008 game application data, and HIPD 1006 can be used as a controller to provide inputs to the game. Similarly, user 1008 can use wrist-wearable device 1002 to initiate a camera of AR glasses 1004, and user 1008 can use wrist-wearable device 1002, AR glasses 1004, and/or HIPD 1006 to manipulate the image capture (e.g., zoom in or out, apply filters, etc.) and capture image data.

Users may interact with the devices disclosed herein in a variety of ways. For example, as shown in FIGS. 11A and 11B, a user 1108 may interact with an AR system 1100 by donning a VR headset 1150 while holding HIPD 1106 and wearing wrist-wearable device 1102. In this example, AR system 1100 may enable a user to interact with a game 1110 by swiping their arm. One or more of VR headset 1150, HIPD 1106, and wrist-wearable device 1102 may detect this gesture and, in response, may display a sword strike in game 1110. Similarly, in FIGS. 12A and 12B, a user 1208 may interact with an AR system 1200 by donning a VR headset 1220 while wearing haptic device 1260 and wrist-wearable device 1230. In this example, AR system 1200 may enable a user to interact with a game 1210 by swiping their arm. One or more of VR headset 1220, haptic device 1260, and wrist-wearable device 1230 may detect this gesture and, in response, may display a spell being cast in game 1110.

Having discussed example AR systems, devices for interacting with such AR systems and other computing systems more generally will now be discussed in greater detail. Some explanations of devices and components that can be included in some or all of the example devices discussed below are explained herein for ease of reference. Certain types of the components described below may be more suitable for a particular set of devices, and less suitable for a different set of devices. But subsequent reference to the components explained here should be considered to be encompassed by the descriptions provided.

In some embodiments discussed below, example devices and systems, including electronic devices and systems, will be addressed. Such example devices and systems are not intended to be limiting, and one of skill in the art will understand that alternative devices and systems to the example devices and systems described herein may be used to perform the operations and construct the systems and devices that are described herein.

An electronic device may be a device that uses electrical energy to perform a specific function. An electronic device can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device may be a device that sits between two other electronic devices and/or a subset of components of one or more electronic devices and facilitates communication, data processing, and/or data transfer between the respective electronic devices and/or electronic components.

An integrated circuit may be an electronic device made up of multiple interconnected electronic components such as transistors, resistors, and capacitors. These components may be etched onto a small piece of semiconductor material, such as silicon. Integrated circuits may include analog integrated circuits, digital integrated circuits, mixed signal integrated circuits, and/or any other suitable type or form of integrated circuit. Examples of integrated circuits include application-specific integrated circuits (ASICs), processing units, central processing units (CPUs), co-processors, and accelerators.

Analog integrated circuits, such as sensors, power management circuits, and operational amplifiers, may process continuous signals and perform analog functions such as amplification, active filtering, demodulation, and mixing. Examples of analog integrated circuits include linear integrated circuits and radio frequency circuits.

Digital integrated circuits, which may be referred to as logic integrated circuits, may include microprocessors, microcontrollers, memory chips, interfaces, power management circuits, programmable devices, and/or any other suitable type or form of integrated circuit. In some embodiments, examples of integrated circuits include central processing units (CPUs),

Processing units, such as CPUs, may be electronic components that are responsible for executing instructions and controlling the operation of an electronic device (e.g., a computer). There are various types of processors that may be used interchangeably, or may be specifically required, by embodiments described herein. For example, a processor may be: (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) an accelerator, such as a graphics processing unit (GPU), designed to accelerate the creation and rendering of images, videos, and animations (e.g., virtual-reality animations, such as three-dimensional modeling); (iv) a field-programmable gate array (FPGA) that can be programmed and reconfigured after manufacturing and/or can be customized to perform specific tasks, such as signal processing, cryptography, and machine learning; and/or (v) a digital signal processor (DSP) designed to perform mathematical operations on signals such as audio, video, and radio waves. One or more processors of one or more electronic devices may be used in various embodiments described herein.

Memory generally refers to electronic components in a computer or electronic device that store data and instructions for the processor to access and manipulate. Examples of memory can include: (i) random access memory (RAM) configured to store data and instructions temporarily; (ii) read-only memory (ROM) configured to store data and instructions permanently (e.g., one or more portions of system firmware, and/or boot loaders) and/or semi-permanently; (iii) flash memory, which can be configured to store data in electronic devices (e.g., USB drives, memory cards, and/or solid-state drives (SSDs)); and/or (iv) cache memory configured to temporarily store frequently accessed data and instructions. Memory, as described herein, can store structured data (e.g., SQL databases, MongoDB databases, GraphQL data, JSON data, etc.). Other examples of data stored in memory can include (i) profile data, including user account data, user settings, and/or other user data stored by the user, (ii) sensor data detected and/or otherwise obtained by one or more sensors, (iii) media content data including stored image data, audio data, documents, and the like, (iv) application data, which can include data collected and/or otherwise obtained and stored during use of an application, and/or any other types of data described herein.

Controllers may be electronic components that manage and coordinate the operation of other components within an electronic device (e.g., controlling inputs, processing data, and/or generating outputs). Examples of controllers can include: (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs.

A power system of an electronic device may be configured to convert incoming electrical power into a form that can be used to operate the device. A power system can include various components, such as (i) a power source, which can be an alternating current (AC) adapter or a direct current (DC) adapter power supply, (ii) a charger input, which can be configured to use a wired and/or wireless connection (which may be part of a peripheral interface, such as a USB, micro-USB interface, near-field magnetic coupling, magnetic inductive and magnetic resonance charging, and/or radio frequency (RF) charging), (iii) a power-management integrated circuit, configured to distribute power to various components of the device and to ensure that the device operates within safe limits (e.g., regulating voltage, controlling current flow, and/or managing heat dissipation), and/or (iv) a battery configured to store power to provide usable power to components of one or more electronic devices.

Peripheral interfaces may be electronic components (e.g., of electronic devices) that allow electronic devices to communicate with other devices or peripherals and can provide the ability to input and output data and signals. Examples of peripheral interfaces can include (i) universal serial bus (USB) and/or micro-USB interfaces configured for connecting devices to an electronic device, (ii) Bluetooth interfaces configured to allow devices to communicate with each other, including Bluetooth low energy (BLE), (iii) near field communication (NFC) interfaces configured to be short-range wireless interfaces for operations such as access control, (iv) POGO pins, which may be small, spring-loaded pins configured to provide a charging interface, (v) wireless charging interfaces, (vi) GPS interfaces, (vii) Wi-Fi interfaces for providing a connection between a device and a wireless network, and/or (viii) sensor interfaces.

Sensors may be electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device), (ii) biopotential-signal sensors, (iii) inertial measurement units (e.g., IMUs) for detecting, for example, angular rate, force, magnetic field, and/or changes in acceleration, (iv) heart rate sensors for measuring a user’s heart rate, (v) SpO2 sensors for measuring blood oxygen saturation and/or other biometric data of a user, (vi) capacitive sensors for detecting changes in potential at a portion of a user’s body (e.g., a sensor-skin interface), and/or (vii) light sensors (e.g., time-of-flight sensors, infrared light sensors, visible light sensors, etc.).

Biopotential-signal-sensing components may be devices used to measure electrical activity within the body (e.g., biopotential-signal sensors). Some types of biopotential-signal sensors include (i) electroencephalography (EEG) sensors configured to measure electrical activity in the brain to diagnose neurological disorders, (ii) electrocardiography (ECG or EKG) sensors configured to measure electrical activity of the heart to diagnose heart problems, (iii) electromyography (EMG) sensors configured to measure the electrical activity of muscles and to diagnose neuromuscular disorders, and (iv) electrooculography (EOG) sensors configure to measure the electrical activity of eye muscles to detect eye movement and diagnose eye disorders.

An application stored in memory of an electronic device (e.g., software) may include instructions stored in the memory. Examples of such applications include (i) games, (ii) word processors, (iii) messaging applications, (iv) media-streaming applications, (v) financial applications, (vi) calendars. (vii) clocks, and (viii) communication interface modules for enabling wired and/or wireless connections between different respective electronic devices (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocols).

A communication interface may be a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, Bluetooth). In some embodiments, a communication interface can refer to a software layer that enables different software programs to communicate with each other (e.g., application programming interfaces (APIs), protocols like HTTP and TCP/IP, etc.).

A graphics module may be a component or software module that is designed to handle graphical operations and/or processes and can include a hardware module and/or a software module.

Non-transitory computer-readable storage media may be physical devices or storage media that can be used to store electronic data in a non-transitory form (e.g., such that the data is stored permanently until it is intentionally deleted or modified).

FIGS. 13 and 14 illustrate an example wrist-wearable device 1300 and an example computer system 1400, in accordance with some embodiments. Wrist-wearable device 1300 is an instance of wearable device 902 described in FIG. 9 herein, such that the wearable device 902 should be understood to have the features of the wrist-wearable device 1300 and vice versa. FIG. 14 illustrates components of the wrist-wearable device 1300, which can be used individually or in combination, including combinations that include other electronic devices and/or electronic components.

FIG. 13 shows a wearable band 1310 and a watch body 1320 (or capsule) being coupled, as discussed below, to form wrist-wearable device 1300. Wrist-wearable device 1300 can perform various functions and/or operations associated with navigating through user interfaces and selectively opening applications as well as the functions and/or operations described above with reference to FIGS. 9-12B.

As will be described in more detail below, operations executed by wrist-wearable device 1300 can include (i) presenting content to a user (e.g., displaying visual content via a display 1305), (ii) detecting (e.g., sensing) user input (e.g., sensing a touch on peripheral button 1323 and/or at a touch screen of the display 1305, a hand gesture detected by sensors (e.g., biopotential sensors)), (iii) sensing biometric data (e.g., neuromuscular signals, heart rate, temperature, sleep, etc.) via one or more sensors 1313, messaging (e.g., text, speech, video, etc.); image capture via one or more imaging devices or cameras 1325, wireless communications (e.g., cellular, near field, Wi-Fi, personal area network, etc.), location determination, financial transactions, providing haptic feedback, providing alarms, providing notifications, providing biometric authentication, providing health monitoring, providing sleep monitoring, etc.

The above-example functions can be executed independently in watch body 1320, independently in wearable band 1310, and/or via an electronic communication between watch body 1320 and wearable band 1310. In some embodiments, functions can be executed on wrist-wearable device 1300 while an AR environment is being presented (e.g., via one of AR systems 900 to 1200). The wearable devices described herein can also be used with other types of AR environments.

Wearable band 1310 can be configured to be worn by a user such that an inner surface of a wearable structure 1311 of wearable band 1310 is in contact with the user’s skin. In this example, when worn by a user, sensors 1313 may contact the user’s skin. In some examples, one or more of sensors 1313 can sense biometric data such as a user’s heart rate, a saturated oxygen level, temperature, sweat level, neuromuscular signals, or a combination thereof. One or more of sensors 1313 can also sense data about a user’s environment including a user’s motion, altitude, location, orientation, gait, acceleration, position, or a combination thereof. In some embodiment, one or more of sensors 1313 can be configured to track a position and/or motion of wearable band 1310. One or more of sensors 1313 can include any of the sensors defined above and/or discussed below with respect to FIG. 13.

One or more of sensors 1313 can be distributed on an inside and/or an outside surface of wearable band 1310. In some embodiments, one or more of sensors 1313 are uniformly spaced along wearable band 1310. Alternatively, in some embodiments, one or more of sensors 1313 are positioned at distinct points along wearable band 1310. As shown in FIG. 13, one or more of sensors 1313 can be the same or distinct. For example, in some embodiments, one or more of sensors 1313 can be shaped as a pill (e.g., sensor 1313a), an oval, a circle a square, an oblong (e.g., sensor 1313c) and/or any other shape that maintains contact with the user’s skin (e.g., such that neuromuscular signal and/or other biometric data can be accurately measured at the user’s skin). In some embodiments, one or more sensors of 1313 are aligned to form pairs of sensors (e.g., for sensing neuromuscular signals based on differential sensing within each respective sensor). For example, sensor 1313b may be aligned with an adjacent sensor to form sensor pair 1314a and sensor 1313d may be aligned with an adjacent sensor to form sensor pair 1314b. In some embodiments, wearable band 1310 does not have a sensor pair. Alternatively, in some embodiments, wearable band 1310 has a predetermined number of sensor pairs (one pair of sensors, three pairs of sensors, four pairs of sensors, six pairs of sensors, sixteen pairs of sensors, etc.).

Wearable band 1310 can include any suitable number of sensors 1313. In some embodiments, the number and arrangement of sensors 1313 depends on the particular application for which wearable band 1310 is used. For instance, wearable band 1310 can be configured as an armband, wristband, or chest-band that include a plurality of sensors 1313 with different number of sensors 1313, a variety of types of individual sensors with the plurality of sensors 1313, and different arrangements for each use case, such as medical use cases as compared to gaming or general day-to-day use cases.

In accordance with some embodiments, wearable band 1310 further includes an electrical ground electrode and a shielding electrode. The electrical ground and shielding electrodes, like the sensors 1313, can be distributed on the inside surface of the wearable band 1310 such that they contact a portion of the user’s skin. For example, the electrical ground and shielding electrodes can be at an inside surface of a coupling mechanism 1316 or an inside surface of a wearable structure 1311. The electrical ground and shielding electrodes can be formed and/or use the same components as sensors 1313. In some embodiments, wearable band 1310 includes more than one electrical ground electrode and more than one shielding electrode.

Sensors 1313 can be formed as part of wearable structure 1311 of wearable band 1310. In some embodiments, sensors 1313 are flush or substantially flush with wearable structure 1311 such that they do not extend beyond the surface of wearable structure 1311. While flush with wearable structure 1311, sensors 1313 are still configured to contact the user’s skin (e.g., via a skin-contacting surface). Alternatively, in some embodiments, sensors 1313 extend beyond wearable structure 1311 a predetermined distance (e.g., 0.1 – 2 mm) to make contact and depress into the user’s skin. In some embodiment, sensors 1313 are coupled to an actuator (not shown) configured to adjust an extension height (e.g., a distance from the surface of wearable structure 1311) of sensors 1313 such that sensors 1313 make contact and depress into the user’s skin. In some embodiments, the actuators adjust the extension height between 0.01 mm – 1.2 mm. This may allow a the user to customize the positioning of sensors 1313 to improve the overall comfort of the wearable band 1310 when worn while still allowing sensors 1313 to contact the user’s skin. In some embodiments, sensors 1313 are indistinguishable from wearable structure 1311 when worn by the user.

Wearable structure 1311 can be formed of an elastic material, elastomers, etc., configured to be stretched and fitted to be worn by the user. In some embodiments, wearable structure 1311 is a textile or woven fabric. As described above, sensors 1313 can be formed as part of a wearable structure 1311. For example, sensors 1313 can be molded into the wearable structure 1311, be integrated into a woven fabric (e.g., sensors 1313 can be sewn into the fabric and mimic the pliability of fabric and can and/or be constructed from a series woven strands of fabric).

Wearable structure 1311 can include flexible electronic connectors that interconnect sensors 1313, the electronic circuitry, and/or other electronic components (described below in reference to FIG. 14) that are enclosed in wearable band 1310. In some embodiments, the flexible electronic connectors are configured to interconnect sensors 1313, the electronic circuitry, and/or other electronic components of wearable band 1310 with respective sensors and/or other electronic components of another electronic device (e.g., watch body 1320). The flexible electronic connectors are configured to move with wearable structure 1311 such that the user adjustment to wearable structure 1311 (e.g., resizing, pulling, folding, etc.) does not stress or strain the electrical coupling of components of wearable band 1310.

As described above, wearable band 1310 is configured to be worn by a user. In particular, wearable band 1310 can be shaped or otherwise manipulated to be worn by a user. For example, wearable band 1310 can be shaped to have a substantially circular shape such that it can be configured to be worn on the user’s lower arm or wrist. Alternatively, wearable band 1310 can be shaped to be worn on another body part of the user, such as the user’s upper arm (e.g., around a bicep), forearm, chest, legs, etc. Wearable band 1310 can include a retaining mechanism 1312 (e.g., a buckle, a hook and loop fastener, etc.) for securing wearable band 1310 to the user’s wrist or other body part. While wearable band 1310 is worn by the user, sensors 1313 sense data (referred to as sensor data) from the user’s skin. In some examples, sensors 1313 of wearable band 1310 obtain (e.g., sense and record) neuromuscular signals.

The sensed data (e.g., sensed neuromuscular signals) can be used to detect and/or determine the user’s intention to perform certain motor actions. In some examples, sensors 1313 may sense and record neuromuscular signals from the user as the user performs muscular activations (e.g., movements, gestures, etc.). The detected and/or determined motor actions (e.g., phalange (or digit) movements, wrist movements, hand movements, and/or other muscle intentions) can be used to determine control commands or control information (instructions to perform certain commands after the data is sensed) for causing a computing device to perform one or more input commands. For example, the sensed neuromuscular signals can be used to control certain user interfaces displayed on display 1305 of wrist-wearable device 1300 and/or can be transmitted to a device responsible for rendering an artificial-reality environment (e.g., a head-mounted display) to perform an action in an associated artificial-reality environment, such as to control the motion of a virtual device displayed to the user. The muscular activations performed by the user can include static gestures, such as placing the user’s hand palm down on a table, dynamic gestures, such as grasping a physical or virtual object, and covert gestures that are imperceptible to another person, such as slightly tensing a joint by co-contracting opposing muscles or using sub-muscular activations. The muscular activations performed by the user can include symbolic gestures (e.g., gestures mapped to other gestures, interactions, or commands, for example, based on a gesture vocabulary that specifies the mapping of gestures to commands).

The sensor data sensed by sensors 1313 can be used to provide a user with an enhanced interaction with a physical object (e.g., devices communicatively coupled with wearable band 1310) and/or a virtual object in an artificial-reality application generated by an artificial-reality system (e.g., user interface objects presented on the display 1305, or another computing device (e.g., a smartphone)).

In some embodiments, wearable band 1310 includes one or more haptic devices 1446 (e.g., a vibratory haptic actuator) that are configured to provide haptic feedback (e.g., a cutaneous and/or kinesthetic sensation, etc.) to the user’s skin. Sensors 1313 and/or haptic devices 1446 (shown in FIG. 14) can be configured to operate in conjunction with multiple applications including, without limitation, health monitoring, social media, games, and artificial reality (e.g., the applications associated with artificial reality).

Wearable band 1310 can also include coupling mechanism 1316 for detachably coupling a capsule (e.g., a computing unit) or watch body 1320 (via a coupling surface of the watch body 1320) to wearable band 1310. For example, a cradle or a shape of coupling mechanism 1316 can correspond to shape of watch body 1320 of wrist-wearable device 1300. In particular, coupling mechanism 1316 can be configured to receive a coupling surface proximate to the bottom side of watch body 1320 (e.g., a side opposite to a front side of watch body 1320 where display 1305 is located), such that a user can push watch body 1320 downward into coupling mechanism 1316 to attach watch body 1320 to coupling mechanism 1316. In some embodiments, coupling mechanism 1316 can be configured to receive a top side of the watch body 1320 (e.g., a side proximate to the front side of watch body 1320 where display 1305 is located) that is pushed upward into the cradle, as opposed to being pushed downward into coupling mechanism 1316. In some embodiments, coupling mechanism 1316 is an integrated component of wearable band 1310 such that wearable band 1310 and coupling mechanism 1316 are a single unitary structure. In some embodiments, coupling mechanism 1316 is a type of frame or shell that allows watch body 1320 coupling surface to be retained within or on wearable band 1310 coupling mechanism 1316 (e.g., a cradle, a tracker band, a support base, a clasp, etc.).

Coupling mechanism 1316 can allow for watch body 1320 to be detachably coupled to the wearable band 1310 through a friction fit, magnetic coupling, a rotation-based connector, a shear-pin coupler, a retention spring, one or more magnets, a clip, a pin shaft, a hook and loop fastener, or a combination thereof. A user can perform any type of motion to couple the watch body 1320 to wearable band 1310 and to decouple the watch body 1320 from the wearable band 1310. For example, a user can twist, slide, turn, push, pull, or rotate watch body 1320 relative to wearable band 1310, or a combination thereof, to attach watch body 1320 to wearable band 1310 and to detach watch body 1320 from wearable band 1310. Alternatively, as discussed below, in some embodiments, the watch body 1320 can be decoupled from the wearable band 1310 by actuation of a release mechanism 1329.

Wearable band 1310 can be coupled with watch body 1320 to increase the functionality of wearable band 1310 (e.g., converting wearable band 1310 into wrist-wearable device 1300, adding an additional computing unit and/or battery to increase computational resources and/or a battery life of wearable band 1310, adding additional sensors to improve sensed data, etc.). As described above, wearable band 1310 and coupling mechanism 1316 are configured to operate independently (e.g., execute functions independently) from watch body 1320. For example, coupling mechanism 1316 can include one or more sensors 1313 that contact a user’s skin when wearable band 1310 is worn by the user, with or without watch body 1320 and can provide sensor data for determining control commands.

A user can detach watch body 1320 from wearable band 1310 to reduce the encumbrance of wrist-wearable device 1300 to the user. For embodiments in which watch body 1320 is removable, watch body 1320 can be referred to as a removable structure, such that in these embodiments wrist-wearable device 1300 includes a wearable portion (e.g., wearable band 1310) and a removable structure (e.g., watch body 1320).

Turning to watch body 1320, in some examples watch body 1320 can have a substantially rectangular or circular shape. Watch body 1320 is configured to be worn by the user on their wrist or on another body part. More specifically, watch body 1320 is sized to be easily carried by the user, attached on a portion of the user’s clothing, and/or coupled to wearable band 1310 (forming the wrist-wearable device 1300). As described above, watch body 1320 can have a shape corresponding to coupling mechanism 1316 of wearable band 1310. In some embodiments, watch body 1320 includes a single release mechanism 1329 or multiple release mechanisms (e.g., two release mechanisms 1329 positioned on opposing sides of watch body 1320, such as spring-loaded buttons) for decoupling watch body 1320 from wearable band 1310. Release mechanism 1329 can include, without limitation, a button, a knob, a plunger, a handle, a lever, a fastener, a clasp, a dial, a latch, or a combination thereof.

A user can actuate release mechanism 1329 by pushing, turning, lifting, depressing, shifting, or performing other actions on release mechanism 1329. Actuation of release mechanism 1329 can release (e.g., decouple) watch body 1320 from coupling mechanism 1316 of wearable band 1310, allowing the user to use watch body 1320 independently from wearable band 1310 and vice versa. For example, decoupling watch body 1320 from wearable band 1310 can allow a user to capture images using rear-facing camera 1325b. Although release mechanism 1329 is shown positioned at a corner of watch body 1320, release mechanism 1329 can be positioned anywhere on watch body 1320 that is convenient for the user to actuate. In addition, in some embodiments, wearable band 1310 can also include a respective release mechanism for decoupling watch body 1320 from coupling mechanism 1316. In some embodiments, release mechanism 1329 is optional and watch body 1320 can be decoupled from coupling mechanism 1316 as described above (e.g., via twisting, rotating, etc.).

Watch body 1320 can include one or more peripheral buttons 1323 and 1327 for performing various operations at watch body 1320. For example, peripheral buttons 1323 and 1327 can be used to turn on or wake (e.g., transition from a sleep state to an active state) display 1305, unlock watch body 1320, increase or decrease a volume, increase or decrease a brightness, interact with one or more applications, interact with one or more user interfaces, etc. Additionally or alternatively, in some embodiments, display 1305 operates as a touch screen and allows the user to provide one or more inputs for interacting with watch body 1320.

In some embodiments, watch body 1320 includes one or more sensors 1321. Sensors 1321 of watch body 1320 can be the same or distinct from sensors 1313 of wearable band 1310. Sensors 1321 of watch body 1320 can be distributed on an inside and/or an outside surface of watch body 1320. In some embodiments, sensors 1321 are configured to contact a user’s skin when watch body 1320 is worn by the user. For example, sensors 1321 can be placed on the bottom side of watch body 1320 and coupling mechanism 1316 can be a cradle with an opening that allows the bottom side of watch body 1320 to directly contact the user’s skin. Alternatively, in some embodiments, watch body 1320 does not include sensors that are configured to contact the user’s skin (e.g., including sensors internal and/or external to the watch body 1320 that are configured to sense data of watch body 1320 and the surrounding environment). In some embodiments, sensors 1321 are configured to track a position and/or motion of watch body 1320.

Watch body 1320 and wearable band 1310 can share data using a wired communication method (e.g., a Universal Asynchronous Receiver/Transmitter (UART), a USB transceiver, etc.) and/or a wireless communication method (e.g., near field communication, Bluetooth, etc.). For example, watch body 1320 and wearable band 1310 can share data sensed by sensors 1313 and 1321, as well as application and device specific information (e.g., active and/or available applications, output devices (e.g., displays, speakers, etc.), input devices (e.g., touch screens, microphones, imaging sensors, etc.).

In some embodiments, watch body 1320 can include, without limitation, a front-facing camera 1325a and/or a rear-facing camera 1325b, sensors 1321 (e.g., a biometric sensor, an IMU, a heart rate sensor, a saturated oxygen sensor, a neuromuscular signal sensor, an altimeter sensor, a temperature sensor, a bioimpedance sensor, a pedometer sensor, an optical sensor (e.g., imaging sensor 1463), a touch sensor, a sweat sensor, etc.). In some embodiments, watch body 1320 can include one or more haptic devices 1476 (e.g., a vibratory haptic actuator) that is configured to provide haptic feedback (e.g., a cutaneous and/or kinesthetic sensation, etc.) to the user. Sensors 1421 and/or haptic device 1476 can also be configured to operate in conjunction with multiple applications including, without limitation, health monitoring applications, social media applications, game applications, and artificial reality applications (e.g., the applications associated with artificial reality).

As described above, watch body 1320 and wearable band 1310, when coupled, can form wrist-wearable device 1300. When coupled, watch body 1320 and wearable band 1310 may operate as a single device to execute functions (operations, detections, communications, etc.) described herein. In some embodiments, each device may be provided with particular instructions for performing the one or more operations of wrist-wearable device 1300. For example, in accordance with a determination that watch body 1320 does not include neuromuscular signal sensors, wearable band 1310 can include alternative instructions for performing associated instructions (e.g., providing sensed neuromuscular signal data to watch body 1320 via a different electronic device). Operations of wrist-wearable device 1300 can be performed by watch body 1320 alone or in conjunction with wearable band 1310 (e.g., via respective processors and/or hardware components) and vice versa. In some embodiments, operations of wrist-wearable device 1300, watch body 1320, and/or wearable band 1310 can be performed in conjunction with one or more processors and/or hardware components.

As described below with reference to the block diagram of FIG. 14, wearable band 1310 and/or watch body 1320 can each include independent resources required to independently execute functions. For example, wearable band 1310 and/or watch body 1320 can each include a power source (e.g., a battery), a memory, data storage, a processor (e.g., a central processing unit (CPU)), communications, a light source, and/or input/output devices.

FIG. 14 shows block diagrams of a computing system 1430 corresponding to wearable band 1310 and a computing system 1460 corresponding to watch body 1320 according to some embodiments. Computing system 1400 of wrist-wearable device 1300 may include a combination of components of wearable band computing system 1430 and watch body computing system 1460, in accordance with some embodiments.

Watch body 1320 and/or wearable band 1310 can include one or more components shown in watch body computing system 1460. In some embodiments, a single integrated circuit may include all or a substantial portion of the components of watch body computing system 1460 included in a single integrated circuit. Alternatively, in some embodiments, components of the watch body computing system 1460 may be included in a plurality of integrated circuits that are communicatively coupled. In some embodiments, watch body computing system 1460 may be configured to couple (e.g., via a wired or wireless connection) with wearable band computing system 1430, which may allow the computing systems to share components, distribute tasks, and/or perform other operations described herein (individually or as a single device).

Watch body computing system 1460 can include one or more processors 1479, a controller 1477, a peripherals interface 1461, a power system 1495, and memory (e.g., a memory 1480).

Power system 1495 can include a charger input 1496, a power-management integrated circuit (PMIC) 1497, and a battery 1498. In some embodiments, a watch body 1320 and a wearable band 1310 can have respective batteries (e.g., battery 1498 and 1459) and can share power with each other. Watch body 1320 and wearable band 1310 can receive a charge using a variety of techniques. In some embodiments, watch body 1320 and wearable band 1310 can use a wired charging assembly (e.g., power cords) to receive the charge. Alternatively, or in addition, watch body 1320 and/or wearable band 1310 can be configured for wireless charging. For example, a portable charging device can be designed to mate with a portion of watch body 1320 and/or wearable band 1310 and wirelessly deliver usable power to battery 1498 of watch body 1320 and/or battery 1459 of wearable band 1310. Watch body 1320 and wearable band 1310 can have independent power systems (e.g., power system 1495 and 1456, respectively) to enable each to operate independently. Watch body 1320 and wearable band 1310 can also share power (e.g., one can charge the other) via respective PMICs (e.g., PMICs 1497 and 1458) and charger inputs (e.g., 1457 and 1496) that can share power over power and ground conductors and/or over wireless charging antennas.

In some embodiments, peripherals interface 1461 can include one or more sensors 1421. Sensors 1421 can include one or more coupling sensors 1462 for detecting when watch body 1320 is coupled with another electronic device (e.g., a wearable band 1310). Sensors 1421 can include one or more imaging sensors 1463 (e.g., one or more of cameras 1425, and/or separate imaging sensors 1463 (e.g., thermal-imaging sensors)). In some embodiments, sensors 1421 can include one or more SpO2 sensors 1464. In some embodiments, sensors 1421 can include one or more biopotential-signal sensors (e.g., EMG sensors 1465, which may be disposed on an interior, user-facing portion of watch body 1320 and/or wearable band 1310). In some embodiments, sensors 1421 may include one or more capacitive sensors 1466. In some embodiments, sensors 1421 may include one or more heart rate sensors 1467. In some embodiments, sensors 1421 may include one or more IMU sensors 1468. In some embodiments, one or more IMU sensors 1468 can be configured to detect movement of a user’s hand or other location where watch body 1320 is placed or held.

In some embodiments, one or more of sensors 1421 may provide an example human-machine interface. For example, a set of neuromuscular sensors, such as EMG sensors 1465, may be arranged circumferentially around wearable band 1310 with an interior surface of EMG sensors 1465 being configured to contact a user’s skin. Any suitable number of neuromuscular sensors may be used (e.g., between 2 and 20 sensors). The number and arrangement of neuromuscular sensors may depend on the particular application for which the wearable device is used. For example, wearable band 1310 can be used to generate control information for controlling an augmented reality system, a robot, controlling a vehicle, scrolling through text, controlling a virtual avatar, or any other suitable control task.

In some embodiments, neuromuscular sensors may be coupled together using flexible electronics incorporated into the wireless device, and the output of one or more of the sensing components can be optionally processed using hardware signal processing circuitry (e.g., to perform amplification, filtering, and/or rectification). In other embodiments, at least some signal processing of the output of the sensing components can be performed in software such as processors 1479. Thus, signal processing of signals sampled by the sensors can be performed in hardware, software, or by any suitable combination of hardware and software, as aspects of the technology described herein are not limited in this respect.

Neuromuscular signals may be processed in a variety of ways. For example, the output of EMG sensors 1465 may be provided to an analog front end, which may be configured to perform analog processing (e.g., amplification, noise reduction, filtering, etc.) on the recorded signals. The processed analog signals may then be provided to an analog-to-digital converter, which may convert the analog signals to digital signals that can be processed by one or more computer processors. Furthermore, although this example is as discussed in the context of interfaces with EMG sensors, the embodiments described herein can also be implemented in wearable interfaces with other types of sensors including, but not limited to, mechanomyography (MMG) sensors, sonomyography (SMG) sensors, and electrical impedance tomography (EIT) sensors.

In some embodiments, peripherals interface 1461 includes a near-field communication (NFC) component 1469, a global-position system (GPS) component 1470, a long-term evolution (LTE) component 1471, and/or a Wi-Fi and/or Bluetooth communication component 1472. In some embodiments, peripherals interface 1461 includes one or more buttons 1473 (e.g., peripheral buttons 1323 and 1327 in FIG. 13), which, when selected by a user, cause operation to be performed at watch body 1320. In some embodiments, the peripherals interface 1461 includes one or more indicators, such as a light emitting diode (LED), to provide a user with visual indicators (e.g., message received, low battery, active microphone and/or camera, etc.).

Watch body 1320 can include at least one display 1305 for displaying visual representations of information or data to a user, including user-interface elements and/or three-dimensional virtual objects. The display can also include a touch screen for inputting user inputs, such as touch gestures, swipe gestures, and the like. Watch body 1320 can include at least one speaker 1474 and at least one microphone 1475 for providing audio signals to the user and receiving audio input from the user. The user can provide user inputs through microphone 1475 and can also receive audio output from speaker 1474 as part of a haptic event provided by haptic controller 1478. Watch body 1320 can include at least one camera 1425, including a front camera 1425a and a rear camera 1425b. Cameras 1425 can include ultra-wide-angle cameras, wide angle cameras, fish-eye cameras, spherical cameras, telephoto cameras, depth-sensing cameras, or other types of cameras.

Watch body computing system 1460 can include one or more haptic controllers 1478 and associated componentry (e.g., haptic devices 1476) for providing haptic events at watch body 1320 (e.g., a vibrating sensation or audio output in response to an event at the watch body 1320). Haptic controllers 1478 can communicate with one or more haptic devices 1476, such as electroacoustic devices, including a speaker of the one or more speakers 1474 and/or other audio components and/or electromechanical devices that convert energy into linear motion such as a motor, solenoid, electroactive polymer, piezoelectric actuator, electrostatic actuator, or other tactile output generating components (e.g., a component that converts electrical signals into tactile outputs on the device). Haptic controller 1478 can provide haptic events to that are capable of being sensed by a user of watch body 1320. In some embodiments, one or more haptic controllers 1478 can receive input signals from an application of applications 1482.

In some embodiments, wearable band computing system 1430 and/or watch body computing system 1460 can include memory 1480, which can be controlled by one or more memory controllers of controllers 1477. In some embodiments, software components stored in memory 1480 include one or more applications 1482 configured to perform operations at the watch body 1320. In some embodiments, one or more applications 1482 may include games, word processors, messaging applications, calling applications, web browsers, social media applications, media streaming applications, financial applications, calendars, clocks, etc. In some embodiments, software components stored in memory 1480 include one or more communication interface modules 1483 as defined above. In some embodiments, software components stored in memory 1480 include one or more graphics modules 1484 for rendering, encoding, and/or decoding audio and/or visual data and one or more data management modules 1485 for collecting, organizing, and/or providing access to data 1487 stored in memory 1480. In some embodiments, one or more of applications 1482 and/or one or more modules can work in conjunction with one another to perform various tasks at the watch body 1320.

In some embodiments, software components stored in memory 1480 can include one or more operating systems 1481 (e.g., a Linux-based operating system, an Android operating system, etc.). Memory 1480 can also include data 1487. Data 1487 can include profile data 1488A, sensor data 1489A, media content data 1490, and application data 1491.

It should be appreciated that watch body computing system 1460 is an example of a computing system within watch body 1320, and that watch body 1320 can have more or fewer components than shown in watch body computing system 1460, can combine two or more components, and/or can have a different configuration and/or arrangement of the components. The various components shown in watch body computing system 1460 are implemented in hardware, software, firmware, or a combination thereof, including one or more signal processing and/or application-specific integrated circuits.

Turning to the wearable band computing system 1430, one or more components that can be included in wearable band 1310 are shown. Wearable band computing system 1430 can include more or fewer components than shown in watch body computing system 1460, can combine two or more components, and/or can have a different configuration and/or arrangement of some or all of the components. In some embodiments, all, or a substantial portion of the components of wearable band computing system 1430 are included in a single integrated circuit. Alternatively, in some embodiments, components of wearable band computing system 1430 are included in a plurality of integrated circuits that are communicatively coupled. As described above, in some embodiments, wearable band computing system 1430 is configured to couple (e.g., via a wired or wireless connection) with watch body computing system 1460, which allows the computing systems to share components, distribute tasks, and/or perform other operations described herein (individually or as a single device).

Wearable band computing system 1430, similar to watch body computing system 1460, can include one or more processors 1449, one or more controllers 1447 (including one or more haptics controllers 1448), a peripherals interface 1431 that can includes one or more sensors 1413 and other peripheral devices, a power source (e.g., a power system 1456), and memory (e.g., a memory 1450) that includes an operating system (e.g., an operating system 1451), data (e.g., data 1454 including profile data 1488B, sensor data 1489B, etc.), and one or more modules (e.g., a communications interface module 1452, a data management module 1453, etc.).

One or more of sensors 1413 can be analogous to sensors 1421 of watch body computing system 1460. For example, sensors 1413 can include one or more coupling sensors 1432, one or more SpO2 sensors 1434, one or more EMG sensors 1435, one or more capacitive sensors 1436, one or more heart rate sensors 1437, and one or more IMU sensors 1438.

Peripherals interface 1431 can also include other components analogous to those included in peripherals interface 1461 of watch body computing system 1460, including an NFC component 1439, a GPS component 1440, an LTE component 1441, a Wi-Fi and/or Bluetooth communication component 1442, and/or one or more haptic devices 1446 as described above in reference to peripherals interface 1461. In some embodiments, peripherals interface 1431 includes one or more buttons 1443, a display 1433, a speaker 1444, a microphone 1445, and a camera 1455. In some embodiments, peripherals interface 1431 includes one or more indicators, such as an LED.

It should be appreciated that wearable band computing system 1430 is an example of a computing system within wearable band 1310, and that wearable band 1310 can have more or fewer components than shown in wearable band computing system 1430, combine two or more components, and/or have a different configuration and/or arrangement of the components. The various components shown in wearable band computing system 1430 can be implemented in one or more of a combination of hardware, software, or firmware, including one or more signal processing and/or application-specific integrated circuits.

Wrist-wearable device 1300 with respect to FIG. 13 is an example of wearable band 1310 and watch body 1320 coupled together, so wrist-wearable device 1300 will be understood to include the components shown and described for wearable band computing system 1430 and watch body computing system 1460. In some embodiments, wrist-wearable device 1300 has a split architecture (e.g., a split mechanical architecture, a split electrical architecture, etc.) between watch body 1320 and wearable band 1310. In other words, all of the components shown in wearable band computing system 1430 and watch body computing system 1460 can be housed or otherwise disposed in a combined wrist-wearable device 1300 or within individual components of watch body 1320, wearable band 1310, and/or portions thereof (e.g., a coupling mechanism 1316 of wearable band 1310).

The techniques described above can be used with any device for sensing neuromuscular signals but could also be used with other types of wearable devices for sensing neuromuscular signals (such as body-wearable or head-wearable devices that might have neuromuscular sensors closer to the brain or spinal column).

In some embodiments, wrist-wearable device 1300 can be used in conjunction with a head-wearable device (e.g., AR glasses 1500 and VR system 1610) and/or an HIPD, and wrist-wearable device 1300 can also be configured to be used to allow a user to control any aspect of the artificial reality (e.g., by using EMG-based gestures to control user interface objects in the artificial reality and/or by allowing a user to interact with the touchscreen on the wrist-wearable device to also control aspects of the artificial reality). Having thus described example wrist-wearable devices, attention will now be turned to example head-wearable devices, such AR glasses 1500 and VR headset 1610.

FIGS. 15-17 show example artificial-reality systems, which can be used as or in connection with wrist-wearable device 1300. In some embodiments, AR system 1500 includes an eyewear device 1502, as shown in FIG. 15. In some embodiments, VR system 1610 includes a head-mounted display (HMD) 1612, as shown in FIGS. 16A and 16B. In some embodiments, AR system 1500 and VR system 1610 can include one or more analogous components (e.g., components for presenting interactive artificial-reality environments, such as processors, memory, and/or presentation devices, including one or more displays and/or one or more waveguides), some of which are described in more detail with respect to FIG. 17. As described herein, a head-wearable device can include components of eyewear device 1502 and/or head-mounted display 1612. Some embodiments of head-wearable devices do not include any displays, including any of the displays described with respect to AR system 1500 and/or VR system 1610. While the example artificial-reality systems are respectively described herein as AR system 1500 and VR system 1610, either or both of the example AR systems described herein can be configured to present fully-immersive virtual-reality scenes presented in substantially all of a user’s field of view or subtler augmented-reality scenes that are presented within a portion, less than all, of the user’s field of view.

FIG. 15 show an example visual depiction of AR system 1500, including an eyewear device 1502 (which may also be described herein as augmented-reality glasses, and/or smart glasses). AR system 1500 can include additional electronic components that are not shown in FIG. 15, such as a wearable accessory device and/or an intermediary processing device, in electronic communication or otherwise configured to be used in conjunction with the eyewear device 1502. In some embodiments, the wearable accessory device and/or the intermediary processing device may be configured to couple with eyewear device 1502 via a coupling mechanism in electronic communication with a coupling sensor 1724 (FIG. 17), where coupling sensor 1724 can detect when an electronic device becomes physically or electronically coupled with eyewear device 1502. In some embodiments, eyewear device 1502 can be configured to couple to a housing 1790 (FIG. 17), which may include one or more additional coupling mechanisms configured to couple with additional accessory devices. The components shown in FIG. 15 can be implemented in hardware, software, firmware, or a combination thereof, including one or more signal-processing components and/or application-specific integrated circuits (ASICs).

Eyewear device 1502 includes mechanical glasses components, including a frame 1504 configured to hold one or more lenses (e.g., one or both lenses 1506-1 and 1506-2). One of ordinary skill in the art will appreciate that eyewear device 1502 can include additional mechanical components, such as hinges configured to allow portions of frame 1504 of eyewear device 1502 to be folded and unfolded, a bridge configured to span the gap between lenses 1506-1 and 1506-2 and rest on the user’s nose, nose pads configured to rest on the bridge of the nose and provide support for eyewear device 1502, earpieces configured to rest on the user’s ears and provide additional support for eyewear device 1502, temple arms configured to extend from the hinges to the earpieces of eyewear device 1502, and the like. One of ordinary skill in the art will further appreciate that some examples of AR system 1500 can include none of the mechanical components described herein. For example, smart contact lenses configured to present artificial reality to users may not include any components of eyewear device 1502.

Eyewear device 1502 includes electronic components, many of which will be described in more detail below with respect to FIG. 10. Some example electronic components are illustrated in FIG. 15, including acoustic sensors 1525-1, 1525-2, 1525-3, 1525-4, 1525-5, and 1525-6, which can be distributed along a substantial portion of the frame 1504 of eyewear device 1502. Eyewear device 1502 also includes a left camera 1539A and a right camera 1539B, which are located on different sides of the frame 1504. Eyewear device 1502 also includes a processor 1548 (or any other suitable type or form of integrated circuit) that is embedded into a portion of the frame 1504.

FIGS. 16A and 16B show a VR system 1610 that includes a head-mounted display (HMD) 1612 (e.g., also referred to herein as an artificial-reality headset, a head-wearable device, a VR headset, etc.), in accordance with some embodiments. As noted, some artificial-reality systems (e.g., AR system 1500) may, instead of blending an artificial reality with actual reality, substantially replace one or more of a user’s visual and/or other sensory perceptions of the real world with a virtual experience (e.g., AR systems 1100 and 1200).

HMD 1612 includes a front body 1614 and a frame 1616 (e.g., a strap or band) shaped to fit around a user’s head. In some embodiments, front body 1614 and/or frame 1616 include one or more electronic elements for facilitating presentation of and/or interactions with an AR and/or VR system (e.g., displays, IMUs, tracking emitter or detectors). In some embodiments, HMD 1612 includes output audio transducers (e.g., an audio transducer 1618), as shown in FIG. 16B. In some embodiments, one or more components, such as the output audio transducer(s) 1618 and frame 1616, can be configured to attach and detach (e.g., are detachably attachable) to HMD 1612 (e.g., a portion or all of frame 1616, and/or audio transducer 1618), as shown in FIG. 16B. In some embodiments, coupling a detachable component to HMD 1612 causes the detachable component to come into electronic communication with HMD 1612.

FIGS. 16A and 16B also show that VR system 1610 includes one or more cameras, such as left camera 1639A and right camera 1639B, which can be analogous to left and right cameras 1539A and 1539B on frame 1504 of eyewear device 1502. In some embodiments, VR system 1610 includes one or more additional cameras (e.g., cameras 1639C and 1639D), which can be configured to augment image data obtained by left and right cameras 1639A and 1639B by providing more information. For example, camera 1639C can be used to supply color information that is not discerned by cameras 1639A and 1639B. In some embodiments, one or more of cameras 1639A to 1639D can include an optional IR cut filter configured to remove IR light from being received at the respective camera sensors.

FIG. 17 illustrates a computing system 1720 and an optional housing 1790, each of which show components that can be included in AR system 1500 and/or VR system 1610. In some embodiments, more or fewer components can be included in optional housing 1790 depending on practical restraints of the respective AR system being described.

In some embodiments, computing system 1720 can include one or more peripherals interfaces 1722A and/or optional housing 1790 can include one or more peripherals interfaces 1722B. Each of computing system 1720 and optional housing 1790 can also include one or more power systems 1742A and 1742B, one or more controllers 1746 (including one or more haptic controllers 1747), one or more processors 1748A and 1748B (as defined above, including any of the examples provided), and memory 1750A and 1750B, which can all be in electronic communication with each other. For example, the one or more processors 1748A and 1748B can be configured to execute instructions stored in memory 1750A and 1750B, which can cause a controller of one or more of controllers 1746 to cause operations to be performed at one or more peripheral devices connected to peripherals interface 1722A and/or 1722B. In some embodiments, each operation described can be powered by electrical power provided by power system 1742A and/or 1742B.

In some embodiments, peripherals interface 1722A can include one or more devices configured to be part of computing system 1720, some of which have been defined above and/or described with respect to the wrist-wearable devices shown in FIGS. 13 and 14. For example, peripherals interface 1722A can include one or more sensors 1723A. Some example sensors 1723A include one or more coupling sensors 1724, one or more acoustic sensors 1725, one or more imaging sensors 1726, one or more EMG sensors 1727, one or more capacitive sensors 1728, one or more IMU sensors 1729, and/or any other types of sensors explained above or described with respect to any other embodiments discussed herein.

In some embodiments, peripherals interfaces 1722A and 1722B can include one or more additional peripheral devices, including one or more NFC devices 1730, one or more GPS devices 1731, one or more LTE devices 1732, one or more Wi-Fi and/or Bluetooth devices 1733, one or more buttons 1734 (e.g., including buttons that are slidable or otherwise adjustable), one or more displays 1735A and 1735B, one or more speakers 1736A and 1736B, one or more microphones 1737, one or more cameras 1738A and 1738B (e.g., including the left camera 1739A and/or a right camera 1739B), one or more haptic devices 1740, and/or any other types of peripheral devices defined above or described with respect to any other embodiments discussed herein.

AR systems can include a variety of types of visual feedback mechanisms (e.g., presentation devices). For example, display devices in AR system 1500 and/or VR system 1610 can include one or more liquid-crystal displays (LCDs), light emitting diode (LED) displays, organic LED (OLED) displays, and/or any other suitable types of display screens. Artificial-reality systems can include a single display screen (e.g., configured to be seen by both eyes), and/or can provide separate display screens for each eye, which can allow for additional flexibility for varifocal adjustments and/or for correcting a refractive error associated with a user’s vision. Some embodiments of AR systems also include optical subsystems having one or more lenses (e.g., conventional concave or convex lenses, Fresnel lenses, or adjustable liquid lenses) through which a user can view a display screen.

For example, respective displays 1735A and 1735B can be coupled to each of the lenses 1506-1 and 1506-2 of AR system 1500. Displays 1735A and 1735B may be coupled to each of lenses 1506-1 and 1506-2, which can act together or independently to present an image or series of images to a user. In some embodiments, AR system 1500 includes a single display 1735A or 1735B (e.g., a near-eye display) or more than two displays 1735A and 1735B. In some embodiments, a first set of one or more displays 1735A and 1735B can be used to present an augmented-reality environment, and a second set of one or more display devices 1735A and 1735B can be used to present a virtual-reality environment. In some embodiments, one or more waveguides are used in conjunction with presenting artificial-reality content to the user of AR system 1500 (e.g., as a means of delivering light from one or more displays 1735A and 1735B to the user’s eyes). In some embodiments, one or more waveguides are fully or partially integrated into the eyewear device 1502. Additionally, or alternatively to display screens, some artificial-reality systems include one or more projection systems. For example, display devices in AR system 1500 and/or VR system 1610 can include micro-LED projectors that project light (e.g., using a waveguide) into display devices, such as clear combiner lenses that allow ambient light to pass through. The display devices can refract the projected light toward a user’s pupil and can enable a user to simultaneously view both artificial-reality content and the real world. Artificial-reality systems can also be configured with any other suitable type or form of image projection system. In some embodiments, one or more waveguides are provided additionally or alternatively to the one or more display(s) 1735A and 1735B.

Computing system 1720 and/or optional housing 1790 of AR system 1500 or VR system 1610 can include some or all of the components of a power system 1742A and 1742B. Power systems 1742A and 1742B can include one or more charger inputs 1743, one or more PMICs 1744, and/or one or more batteries 1745A and 1744B.

Memory 1750A and 1750B may include instructions and data, some or all of which may be stored as non-transitory computer-readable storage media within the memories 1750A and 1750B. For example, memory 1750A and 1750B can include one or more operating systems 1751, one or more applications 1752, one or more communication interface applications 1753A and 1753B, one or more graphics applications 1754A and 1754B, one or more AR processing applications 1755A and 1755B, and/or any other types of data defined above or described with respect to any other embodiments discussed herein.

Memory 1750A and 1750B also include data 1760A and 1760B, which can be used in conjunction with one or more of the applications discussed above. Data 1760A and 1760B can include profile data 1761, sensor data 1762A and 1762B, media content data 1763A, AR application data 1764A and 1764B, and/or any other types of data defined above or described with respect to any other embodiments discussed herein.

In some embodiments, controller 1746 of eyewear device 1502 may process information generated by sensors 1723A and/or 1723B on eyewear device 1502 and/or another electronic device within AR system 1500. For example, controller 1746 can process information from acoustic sensors 1525-1 and 1525-2. For each detected sound, controller 1746 can perform a direction of arrival (DOA) estimation to estimate a direction from which the detected sound arrived at eyewear device 1502 of R system 1500. As one or more of acoustic sensors 1725 (e.g., the acoustic sensors 1525-1, 1525-2) detects sounds, controller 1746 can populate an audio data set with the information (e.g., represented in FIG. 10 as sensor data 1762A and 1762B).

In some embodiments, a physical electronic connector can convey information between eyewear device 1502 and another electronic device and/or between one or more processors 1548, 1748A, 1748B of AR system 1500 or VR system 1610 and controller 1746. The information can be in the form of optical data, electrical data, wireless data, or any other transmittable data form. Moving the processing of information generated by eyewear device 1502 to an intermediary processing device can reduce weight and heat in the eyewear device, making it more comfortable and safer for a user. In some embodiments, an optional wearable accessory device (e.g., an electronic neckband) is coupled to eyewear device 1502 via one or more connectors. The connectors can be wired or wireless connectors and can include electrical and/or non-electrical (e.g., structural) components. In some embodiments, eyewear device 1502 and the wearable accessory device can operate independently without any wired or wireless connection between them.

In some situations, pairing external devices, such as an intermediary processing device (e.g., HIPD 906, 1006, 1106) with eyewear device 1502 (e.g., as part of AR system 1500) enables eyewear device 1502 to achieve a similar form factor of a pair of glasses while still providing sufficient battery and computation power for expanded capabilities. Some, or all, of the battery power, computational resources, and/or additional features of AR system 1500 can be provided by a paired device or shared between a paired device and eyewear device 1502, thus reducing the weight, heat profile, and form factor of eyewear device 1502 overall while allowing eyewear device 1502 to retain its desired functionality. For example, the wearable accessory device can allow components that would otherwise be included on eyewear device 1502 to be included in the wearable accessory device and/or intermediary processing device, thereby shifting a weight load from the user’s head and neck to one or more other portions of the user’s body. In some embodiments, the intermediary processing device has a larger surface area over which to diffuse and disperse heat to the ambient environment. Thus, the intermediary processing device can allow for greater battery and computation capacity than might otherwise have been possible on eyewear device 1502 standing alone. Because weight carried in the wearable accessory device can be less invasive to a user than weight carried in the eyewear device 1502, a user may tolerate wearing a lighter eyewear device and carrying or wearing the paired device for greater lengths of time than the user would tolerate wearing a heavier eyewear device standing alone, thereby enabling an artificial-reality environment to be incorporated more fully into a user’s day-to-day activities.

AR systems can include various types of computer vision components and subsystems. For example, AR system 1500 and/or VR system 1610 can include one or more optical sensors such as two-dimensional (2D) or three-dimensional (3D) cameras, time-of-flight depth sensors, structured light transmitters and detectors, single-beam or sweeping laser rangefinders, 3D LiDAR sensors, and/or any other suitable type or form of optical sensor. An AR system can process data from one or more of these sensors to identify a location of a user and/or aspects of the use’s real-world physical surroundings, including the locations of real-world objects within the real-world physical surroundings. In some embodiments, the methods described herein are used to map the real world, to provide a user with context about real-world surroundings, and/or to generate digital twins (e.g., interactable virtual objects), among a variety of other functions. For example, FIGS. 16A and 16B show VR system 1610 having cameras 1639A to 1639D, which can be used to provide depth information for creating a voxel field and a two-dimensional mesh to provide object information to the user to avoid collisions.

In some embodiments, AR system 1500 and/or VR system 1610 can include haptic (tactile) feedback systems, which may be incorporated into headwear, gloves, body suits, handheld controllers, environmental devices (e.g., chairs or floormats), and/or any other type of device or system, such as the wearable devices discussed herein. The haptic feedback systems may provide various types of cutaneous feedback, including vibration, force, traction, shear, texture, and/or temperature. The haptic feedback systems may also provide various types of kinesthetic feedback, such as motion and compliance. The haptic feedback may be implemented using motors, piezoelectric actuators, fluidic systems, and/or a variety of other types of feedback mechanisms. The haptic feedback systems may be implemented independently of other artificial-reality devices, within other artificial-reality devices, and/or in conjunction with other artificial-reality devices.

In some embodiments of an artificial reality system, such as AR system 1500 and/or VR system 1610, ambient light (e.g., a live feed of the surrounding environment that a user would normally see) can be passed through a display element of a respective head-wearable device presenting aspects of the AR system. In some embodiments, ambient light can be passed through a portion less that is less than all of an AR environment presented within a user’s field of view (e.g., a portion of the AR environment co-located with a physical object in the user’s real-world environment that is within a designated boundary (e.g., a guardian boundary) configured to be used by the user while they are interacting with the AR environment). For example, a visual user interface element (e.g., a notification user interface element) can be presented at the head-wearable device, and an amount of ambient light (e.g., 15-50% of the ambient light) can be passed through the user interface element such that the user can distinguish at least a portion of the physical environment over which the user interface element is being displayed.

In some embodiments, the systems described herein may also include an eye-tracking subsystem designed to identify and track various characteristics of a user’s eye(s), such as the user’s gaze direction. The phrase “eye tracking” may, in some examples, refer to a process by which the position, orientation, and/or motion of an eye is measured, detected, sensed, determined, and/or monitored. The disclosed systems may measure the position, orientation, and/or motion of an eye in a variety of different ways, including through the use of various optical-based eye-tracking techniques, ultrasound-based eye-tracking techniques, etc. An eye-tracking subsystem may be configured in a number of different ways and may include a variety of different eye-tracking hardware components or other computer-vision components. For example, an eye-tracking subsystem may include a variety of different optical sensors, such as two-dimensional (2D) or 3D cameras, time-of-flight depth sensors, single-beam or sweeping laser rangefinders, 3D LiDAR sensors, and/or any other suitable type or form of optical sensor. In this example, a processing subsystem may process data from one or more of these sensors to measure, detect, determine, and/or otherwise monitor the position, orientation, and/or motion of the user’s eye(s).

FIG. 18 is an illustration of an example system 1800 that incorporates an eye-tracking subsystem capable of tracking a user’s eye(s). As depicted in FIG. 18, system 1800 may include a light source 1802, an optical subsystem 1804, an eye-tracking subsystem 1806, and/or a control subsystem 1808. In some examples, light source 1802 may generate light for an image (e.g., to be presented to an eye 1801 of the viewer). Light source 1802 may represent any of a variety of suitable devices. For example, light source 1802 can include a two-dimensional projector (e.g., a LCoS display), a scanning source (e.g., a scanning laser), or other device (e.g., an LCD, an LED display, an OLED display, an active-matrix OLED display (AMOLED), a transparent OLED display (TOLED), a waveguide, or some other display capable of generating light for presenting an image to the viewer). In some examples, the image may represent a virtual image, which may refer to an optical image formed from the apparent divergence of light rays from a point in space, as opposed to an image formed from the light ray’s actual divergence.

In some embodiments, optical subsystem 1804 may receive the light generated by light source 1802 and generate, based on the received light, converging light 1820 that includes the image. In some examples, optical subsystem 1804 may include any number of lenses (e.g., Fresnel lenses, convex lenses, concave lenses), apertures, filters, mirrors, prisms, and/or other optical components, possibly in combination with actuators and/or other devices. In particular, the actuators and/or other devices may translate and/or rotate one or more of the optical components to alter one or more aspects of converging light 1820. Further, various mechanical couplings may serve to maintain the relative spacing and/or the orientation of the optical components in any suitable combination.

In one embodiment, eye-tracking subsystem 1806 may generate tracking information indicating a gaze angle of an eye 1801 of the viewer. In this embodiment, control subsystem 1808 may control aspects of optical subsystem 1804 (e.g., the angle of incidence of converging light 1820) based at least in part on this tracking information. Additionally, in some examples, control subsystem 1808 may store and utilize historical tracking information (e.g., a history of the tracking information over a given duration, such as the previous second or fraction thereof) to anticipate the gaze angle of eye 1801 (e.g., an angle between the visual axis and the anatomical axis of eye 1801). In some embodiments, eye-tracking subsystem 1806 may detect radiation emanating from some portion of eye 1801 (e.g., the cornea, the iris, the pupil, or the like) to determine the current gaze angle of eye 1801. In other examples, eye-tracking subsystem 1806 may employ a wavefront sensor to track the current location of the pupil.

Any number of techniques can be used to track eye 1801. Some techniques may involve illuminating eye 1801 with infrared light and measuring reflections with at least one optical sensor that is tuned to be sensitive to the infrared light. Information about how the infrared light is reflected from eye 1801 may be analyzed to determine the position(s), orientation(s), and/or motion(s) of one or more eye feature(s), such as the cornea, pupil, iris, and/or retinal blood vessels.

In some examples, the radiation captured by a sensor of eye-tracking subsystem 1806 may be digitized (i.e., converted to an electronic signal). Further, the sensor may transmit a digital representation of this electronic signal to one or more processors (for example, processors associated with a device including eye-tracking subsystem 1806). Eye-tracking subsystem 1806 may include any of a variety of sensors in a variety of different configurations. For example, eye-tracking subsystem 1806 may include an infrared detector that reacts to infrared radiation. The infrared detector may be a thermal detector, a photonic detector, and/or any other suitable type of detector. Thermal detectors may include detectors that react to thermal effects of the incident infrared radiation.

In some examples, one or more processors may process the digital representation generated by the sensor(s) of eye-tracking subsystem 1806 to track the movement of eye 1801. In another example, these processors may track the movements of eye 1801 by executing algorithms represented by computer-executable instructions stored on non-transitory memory. In some examples, on-chip logic (e.g., an application-specific integrated circuit or ASIC) may be used to perform at least portions of such algorithms. As noted, eye-tracking subsystem 1806 may be programmed to use an output of the sensor(s) to track movement of eye 1801. In some embodiments, eye-tracking subsystem 1806 may analyze the digital representation generated by the sensors to extract eye rotation information from changes in reflections. In one embodiment, eye-tracking subsystem 1806 may use corneal reflections or glints (also known as Purkinje images) and/or the center of the eye’s pupil 1822 as features to track over time.

In some embodiments, eye-tracking subsystem 1806 may use the center of the eye’s pupil 1822 and infrared or near-infrared, non-collimated light to create corneal reflections. In these embodiments, eye-tracking subsystem 1806 may use the vector between the center of the eye’s pupil 1822 and the corneal reflections to compute the gaze direction of eye 1801. In some embodiments, the disclosed systems may perform a calibration procedure for an individual (using, e.g., supervised or unsupervised techniques) before tracking the user’s eyes. For example, the calibration procedure may include directing users to look at one or more points displayed on a display while the eye-tracking system records the values that correspond to each gaze position associated with each point.

In some embodiments, eye-tracking subsystem 1806 may use two types of infrared and/or near-infrared (also known as active light) eye-tracking techniques: bright-pupil and dark-pupil eye tracking, which may be differentiated based on the location of an illumination source with respect to the optical elements used. If the illumination is coaxial with the optical path, then eye 1801 may act as a retroreflector as the light reflects off the retina, thereby creating a bright pupil effect similar to a red-eye effect in photography. If the illumination source is offset from the optical path, then the eye’s pupil 1822 may appear dark because the retroreflection from the retina is directed away from the sensor. In some embodiments, bright-pupil tracking may create greater iris/pupil contrast, allowing more robust eye tracking with iris pigmentation, and may feature reduced interference (e.g., interference caused by eyelashes and other obscuring features). Bright-pupil tracking may also allow tracking in lighting conditions ranging from total darkness to a very bright environment.

In some embodiments, control subsystem 1808 may control light source 1802 and/or optical subsystem 1804 to reduce optical aberrations (e.g., chromatic aberrations and/or monochromatic aberrations) of the image that may be caused by or influenced by eye 1801. In some examples, as mentioned above, control subsystem 1808 may use the tracking information from eye-tracking subsystem 1806 to perform such control. For example, in controlling light source 1802, control subsystem 1808 may alter the light generated by light source 1802 (e.g., by way of image rendering) to modify (e.g., pre-distort) the image so that the aberration of the image caused by eye 1801 is reduced.

The disclosed systems may track both the position and relative size of the pupil (since, e.g., the pupil dilates and/or contracts). In some examples, the eye-tracking devices and components (e.g., sensors and/or sources) used for detecting and/or tracking the pupil may be different (or calibrated differently) for different types of eyes. For example, the frequency range of the sensors may be different (or separately calibrated) for eyes of different colors and/or different pupil types, sizes, and/or the like. As such, the various eye-tracking components (e.g., infrared sources and/or sensors) described herein may need to be calibrated for each individual user and/or eye.

The disclosed systems may track both eyes with and without ophthalmic correction, such as that provided by contact lenses worn by the user. In some embodiments, ophthalmic correction elements (e.g., adjustable lenses) may be directly incorporated into the artificial reality systems described herein. In some examples, the color of the user’s eye may necessitate modification of a corresponding eye-tracking algorithm. For example, eye-tracking algorithms may need to be modified based at least in part on the differing color contrast between a brown eye and, for example, a blue eye.

FIG. 19 is a more detailed illustration of various aspects of the eye-tracking subsystem illustrated in FIG. 18. As shown in this figure, an eye-tracking subsystem 1900 may include at least one source 1904 and at least one sensor 1906. Source 1904 generally represents any type or form of element capable of emitting radiation. In one example, source 1904 may generate visible, infrared, and/or near-infrared radiation. In some examples, source 1904 may radiate non-collimated infrared and/or near-infrared portions of the electromagnetic spectrum towards an eye 1902 of a user. Source 1904 may utilize a variety of sampling rates and speeds. For example, the disclosed systems may use sources with higher sampling rates in order to capture fixational eye movements of a user’s eye 1902 and/or to correctly measure saccade dynamics of the user’s eye 1902. As noted above, any type or form of eye-tracking technique may be used to track the user’s eye 1902, including optical-based eye-tracking techniques, ultrasound-based eye-tracking techniques, etc.

Sensor 1906 generally represents any type or form of element capable of detecting radiation, such as radiation reflected off the user’s eye 1902. Examples of sensor 1906 include, without limitation, a charge coupled device (CCD), a photodiode array, a complementary metal-oxide-semiconductor (CMOS) based sensor device, and/or the like. In one example, sensor 1906 may represent a sensor having predetermined parameters, including, but not limited to, a dynamic resolution range, linearity, and/or other characteristic selected and/or designed specifically for eye tracking.

As detailed above, eye-tracking subsystem 1900 may generate one or more glints. As detailed above, a glint 1903 may represent reflections of radiation (e.g., infrared radiation from an infrared source, such as source 1904) from the structure of the user’s eye. In various embodiments, glint 1903 and/or the user’s pupil may be tracked using an eye-tracking algorithm executed by a processor (either within or external to an artificial reality device). For example, an artificial reality device may include a processor and/or a memory device in order to perform eye tracking locally and/or a transceiver to send and receive the data necessary to perform eye tracking on an external device (e.g., a mobile phone, cloud server, or other computing device).

FIG. 19 shows an example image 1905 captured by an eye-tracking subsystem, such as eye-tracking subsystem 1900. In this example, image 1905 may include both the user’s pupil 1908 and a glint 1910 near the same. In some examples, pupil 1908 and/or glint 1910 may be identified using an artificial-intelligence-based algorithm, such as a computer-vision-based algorithm. In one embodiment, image 1905 may represent a single frame in a series of frames that may be analyzed continuously in order to track the eye 1902 of the user. Further, pupil 1908 and/or glint 1910 may be tracked over a period of time to determine a user’s gaze.

In one example, eye-tracking subsystem 1900 may be configured to identify and measure the inter-pupillary distance (IPD) of a user. In some embodiments, eye-tracking subsystem 1900 may measure and/or calculate the IPD of the user while the user is wearing the artificial reality system. In these embodiments, eye-tracking subsystem 1900 may detect the positions of a user’s eyes and may use this information to calculate the user’s IPD.

As noted, the eye-tracking systems or subsystems disclosed herein may track a user’s eye position and/or eye movement in a variety of ways. In one example, one or more light sources and/or optical sensors may capture an image of the user’s eyes. The eye-tracking subsystem may then use the captured information to determine the user’s inter-pupillary distance, interocular distance, and/or a 3D position of each eye (e.g., for distortion adjustment purposes), including a magnitude of torsion and rotation (i.e., roll, pitch, and yaw) and/or gaze directions for each eye. In one example, infrared light may be emitted by the eye-tracking subsystem and reflected from each eye. The reflected light may be received or detected by an optical sensor and analyzed to extract eye rotation data from changes in the infrared light reflected by each eye.

The eye-tracking subsystem may use any of a variety of different methods to track the eyes of a user. For example, a light source (e.g., infrared light-emitting diodes) may emit a dot pattern onto each eye of the user. The eye-tracking subsystem may then detect (e.g., via an optical sensor coupled to the artificial reality system) and analyze a reflection of the dot pattern from each eye of the user to identify a location of each pupil of the user. Accordingly, the eye-tracking subsystem may track up to six degrees of freedom of each eye (i.e., 3D position, roll, pitch, and yaw) and at least a subset of the tracked quantities may be combined from two eyes of a user to estimate a gaze point (i.e., a 3D location or position in a virtual scene where the user is looking) and/or an IPD.

In some cases, the distance between a user’s pupil and a display may change as the user’s eye moves to look in different directions. The varying distance between a pupil and a display as viewing direction changes may be referred to as “pupil swim” and may contribute to distortion perceived by the user as a result of light focusing in different locations as the distance between the pupil and the display changes. Accordingly, measuring distortion at different eye positions and pupil distances relative to displays and generating distortion corrections for different positions and distances may allow mitigation of distortion caused by pupil swim by tracking the 3D position of a user’s eyes and applying a distortion correction corresponding to the 3D position of each of the user’s eyes at a given point in time. Thus, knowing the 3D position of each of a user’s eyes may allow for the mitigation of distortion caused by changes in the distance between the pupil of the eye and the display by applying a distortion correction for each 3D eye position. Furthermore, as noted above, knowing the position of each of the user’s eyes may also enable the eye-tracking subsystem to make automated adjustments for a user’s IPD.

In some embodiments, a display subsystem may include a variety of additional subsystems that may work in conjunction with the eye-tracking subsystems described herein. For example, a display subsystem may include a varifocal subsystem, a scene-rendering module, and/or a vergence-processing module. The varifocal subsystem may cause left and right display elements to vary the focal distance of the display device. In one embodiment, the varifocal subsystem may physically change the distance between a display and the optics through which it is viewed by moving the display, the optics, or both. Additionally, moving or translating two lenses relative to each other may also be used to change the focal distance of the display. Thus, the varifocal subsystem may include actuators or motors that move displays and/or optics to change the distance between them. This varifocal subsystem may be separate from or integrated into the display subsystem. The varifocal subsystem may also be integrated into or separate from its actuation subsystem and/or the eye-tracking subsystems described herein.

In one example, the display subsystem may include a vergence-processing module configured to determine a vergence depth of a user’s gaze based on a gaze point and/or an estimated intersection of the gaze lines determined by the eye-tracking subsystem. Vergence may refer to the simultaneous movement or rotation of both eyes in opposite directions to maintain single binocular vision, which may be naturally and automatically performed by the human eye. Thus, a location where a user’s eyes are verged is where the user is looking and is also typically the location where the user’s eyes are focused. For example, the vergence-processing module may triangulate gaze lines to estimate a distance or depth from the user associated with intersection of the gaze lines. The depth associated with intersection of the gaze lines may then be used as an approximation for the accommodation distance, which may identify a distance from the user where the user’s eyes are directed. Thus, the vergence distance may allow for the determination of a location where the user’s eyes should be focused and a depth from the user’s eyes at which the eyes are focused, thereby providing information (such as an object or plane of focus) for rendering adjustments to the virtual scene.

The vergence-processing module may coordinate with the eye-tracking subsystems described herein to make adjustments to the display subsystem to account for a user’s vergence depth. When the user is focused on something at a distance, the user’s pupils may be slightly farther apart than when the user is focused on something close. The eye-tracking subsystem may obtain information about the user’s vergence or focus depth and may adjust the display subsystem to be closer together when the user’s eyes focus or verge on something close and to be farther apart when the user’s eyes focus or verge on something at a distance.

The eye-tracking information generated by the above-described eye-tracking subsystems may also be used, for example, to modify various aspect of how different computer-generated images are presented. For example, a display subsystem may be configured to modify, based on information generated by an eye-tracking subsystem, at least one aspect of how the computer-generated images are presented. For instance, the computer-generated images may be modified based on the user’s eye movement, such that if a user is looking up, the computer-generated images may be moved upward on the screen. Similarly, if the user is looking to the side or down, the computer-generated images may be moved to the side or downward on the screen. If the user’s eyes are closed, the computer-generated images may be paused or removed from the display and resumed once the user’s eyes are back open.

The above-described eye-tracking subsystems can be incorporated into one or more of the various artificial reality systems described herein in a variety of ways. For example, one or more of the various components of system 1800 and/or eye-tracking subsystem 1900 may be incorporated into any of the augmented-reality systems in and/or virtual-reality systems described herein in to enable these systems to perform various eye-tracking tasks (including one or more of the eye-tracking operations described herein).

As detailed above, the computing devices and systems described and/or illustrated herein broadly represent any type or form of computing device or system capable of executing computer-readable instructions, such as those contained within the modules described herein. In their most basic configuration, these computing device(s) may each include at least one memory device and at least one physical processor.

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. For example, one or more of the modules recited herein may receive sensor data to be transformed, transform the sensor data, output a result of the transformation to measure changes over time, use the result of the transformation to determine biometric measurements, and store the result of the transformation to monitor biometric parameters. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

The preceding description has been provided to enable others skilled in the art to best utilize various aspects of the example embodiments disclosed herein. This example description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A device comprising:
an optical sensor comprising a polarizer, wherein the optical sensor is configured to:
capture a polarized light from a skin;
detect a diffused light component from the captured polarized light using the polarizer; and
perform a biometric measurement based on the detected diffused light component.

2. The device of claim 1, wherein the biometric measurement corresponds to at least one of:
analyzing blood volume changes;
monitoring cardiovascular parameters;
photoplethysmography (PPG);
heart rate measurement;
blood oxygen measurement; or
breathing rate measurement.

3. The device of claim 1, further comprising a polarized light source configured to provide linearly polarized light or circularly polarized light to the skin.

4. The device of claim 1, wherein the polarizer comprises at least one of:
a linear polarizer;
an orthogonal polarizer;
a circular polarizer; or
a plurality of linear polarizers.

5. The device of claim 4, wherein the plurality of linear polarizers includes one or more of a 0-degree polarizer, a 45-degree polarizer, a 90-degree polarizer, and a 135-degree polarizer.

6. The device of claim 1, wherein the optical sensor is further configured to:
detect a specular light component from the captured polarized light using the polarizer; and
perform the biometric measurement based on the detected specular light component.

7. The device of claim 6, wherein the biometric measurement based on the captured specular light corresponds to at least one of:
analyzing polarization state changes;
determining skin moisture levels;
monitoring skin hydration;
monitoring sweat rate; or
detecting an oiliness of the skin.

8. The device of claim 1, wherein the optical sensor is configured for dual color detection.

9. The device of claim 1, wherein the optical sensor comprises at least one of:
a point detector;
a scanning system; or
an array of detectors.

10. The device of claim 1, wherein the device comprises a smart glasses form factor and the optical sensor comprises at least one of:
an eye tracking camera; or
a face tracking camera.

11. The device of claim 1, wherein the device comprises a wrist wearable device form factor and the optical sensor comprises a camera for gesture detection.

12. The device of claim 1, wherein the optical sensor is configured to measure a same patch of the skin over time.

13. The device of claim 1, wherein the optical sensor is configured to analyze an intensity of a fluctuation of the diffused light component with respect to a baseline.

14. The device of claim 13, wherein
a high amplitude relative to the baseline indicates an increase in a blood vessel diameter and a low amplitude relative to the baseline indicates a decrease in the blood vessel diameter.

15. A system comprising:
a polarized light source configured to provide linearly polarized light or circularly polarized light to a skin; and
an optical sensor comprising a polarizer, wherein the optical sensor is configured to:
capture a polarized light from the skin;
detect a diffused light component from the captured polarized light using the polarizer; and
perform a biometric measurement based on the detected diffused light component.

16. The system of claim 15, wherein the biometric measurement corresponds to at least one of:
analyzing blood volume changes;
monitoring cardiovascular parameters;
photoplethysmography (PPG);
heart rate measurement;
blood oxygen measurement; or
breathing rate measurement.

17. The system of claim 15, wherein:
the optical sensor is further configured to:
detect a specular light component from the captured polarized light using the polarizer; and
perform the biometric measurement based on the detected specular light component; and
the biometric measurement based on the detected specular light component corresponds to at least one of:
analyzing polarization state changes;
determining skin moisture levels;
monitoring skin hydration;
monitoring sweat rate; or
detecting an oiliness of the skin.

18. The system of claim 15, wherein:
the polarizer comprises at least one of:
a linear polarizer;
an orthogonal polarizer;
a circular polarizer; or
a plurality of linear polarizers that includes at least one of a 0-degree polarizer, a 45-degree polarizer, a 90-degree polarizer, and a 135-degree polarizer; and
the optical sensor comprises at least one of:
a dual color detector;
a point detector;
a scanning system; or
an array of detectors.

19. A method comprising:
capturing a light reflected from a skin;
detecting a polarized light signal from the captured reflected light;
analyzing the detected polarized light signal; and
determining a biometric measurement based on the analysis.

20. The method of claim 19, further comprising:
detecting a specular light component or a diffused light component from the polarized light signal; and
determining the biometric measurement based on the specular light component or the diffused light component.
